(19) United States
(12) Patent Application Publication
(10) Pub. No.: US 2026/0335415 A1
(43) Pub. Date: Oct. 29, 2026

(54) DOSING REGIMEN OF PEGYLATED HEMOGLOBIN

(71) Applicant: Prolong Pharmaceuticals, LLC, South Plainfield, NJ (US)

(72) Inventors: Shaunak G. Pandya, Fogelsville, PA (US); Ronald Jubin, Watchung, NJ (US)

(21) Appl. No.: 19/702,854

(22) Filed: Jun. 9, 2026

Related U.S. Application Data
(63) Continuation of application No. PCT/US2025/025245, filed on Apr. 17, 2025.
(60) Provisional application No. 63/635,099, filed on Apr. 17, 2024.

(51) Int. Cl. A61K 38/42; A61K 47/60; A61P 7/00; A61P 9/10

(52) U.S. Cl. CPC A61K 38/42; A61K 47/60; A61P 7/00; A61P 9/10

(57) ABSTRACT

The present invention discloses an improved infusion process by infusing an adequate amount of PEGylated hemoglobin into a subject in need thereof in at least two cycles. The present invention discloses an improved infusion process of reducing the incidence and/or alleviating one or more symptoms characterized by the lack of blood supply in a subject, by administering PEG-HB-CO in at least two cycles. The present invention also maintains adequate flow rate and blood volume for prevention, amelioration, or treatment of any side effect associated with improper perfusion or perfused in single cycle.

### CROSS-REFERENCE TO RELATED APPLICATIONS

This application is a continuation of International Application No. PCT/US2025/025245, filed on Apr. 17, 2025, which claims the benefit of and priority to U.S. Provisional Application No. 63/635,099, filed on Apr. 17, 2024, which is incorporated herein by reference in its entirety.

### FIELD OF THE INVENTION

The present invention discloses an improved infusion process by infusing an adequate amount of PEGylated hemoglobin into a subject in need thereof in at least two cycles. The present invention discloses an improved infusion process of reducing the incidence and/or alleviating one or more symptoms characterized by the lack of blood supply in a subject, by administering PEG-HB-CO in at least two cycles. The present invention also maintains adequate flow rate and blood volume for improving the tissue oxygenation, and controlling adverse effect of hypervolemia in the subject. The invention maintains the blood pressure in the subject during infusion cycle.

### BACKGROUND OF THE INVENTION

Trauma is a leading cause of death worldwide, and almost 30% of trauma deaths are due to blood loss. The primary reason for the high mortality rate is the inability to maintain tissue oxygenation of the subject between the time of injury and time of surgery at a medical facility. The lack of oxygenation results in tissue damage, organ failure and death. Therefore, a major focus in treating traumatic shock is administering therapeutics providing as much oxygen as possible to internal tissues and organs of the subject.

The present applicant has solved this problem by developing a novel oxygenated pegylated hemoglobin carrying and delivering hemoglobin molecules that can serve as blood substitutes.

However, the applicant noticed the infusion of pegylated hemoglobin is very challenging. Adding a PEGylated hemoglobin (PEG-Hb) or PEGylated carbon monoxide containing hemoglobin (PEG-Hb-CO) infusion to an intravenous fluid line, on proximal occlusion may lead to back-flow into the main line and later administration of a potentially dangerous dose or drug interaction. If the infusion of the PEGylated hemoglobin does not monitor it can lead to hypervolemia which leads to fluid imbalance or can cause for too much fluid in the body. Fluid overload also leads to increase in extracellular fluid (ECF) which is present in the blood and lymph, between the cells, surrounding the brain and spinal cord, inside muscles and tissues, and everywhere else there is space outside of cells, it will filling up body cavities and all the spaces between cells this results in symptoms such as swelling, rapid weight gain, high blood pressure, cardiovascular events, organ damage by pressure, and shortness of breath.

Free (Hb) outside of the red blood cell is physiologically unstable and can promote serious effects including vascular patency and interstitial tissue inflammation. Free Hb in circulation is readily sequestered by haptoglobin, hemopexin and other factors to eliminate the protein before it can promote significant injury and inflammation. Early attempts to use free Hb products failed for poor pharmacokinetics and toxicity. Treating with very high Hb levels to overcome poor PK did not provide sustained benefits and instead overwhelmed the Hb clearance mechanisms and extravasating into the interstitial space. These free Hb and degradants would bind and sequester nitric oxide promoting vessel constriction as well as promoting inflammation. Further efforts to overcome effects of free Hb included approaches to increase the molecular weight thereby reduce rapid clearance and extravasation. These included agents modified by cross-linking into various dimeric, trimeric or tetrameric versions.

These agents also showed toxicity when often these modifications prevented haptoglobin and/or hemopexin binding that promote normal clearance pathways. PEG-Hb-CO is uniquely designed through a PEGylation process that increases its molecular size preventing extravasation but retains haptoglobin binding and clearance similar to non-modified. Therefore, PEG-Hb-CO does not exhibit vasoconstrictive or inflammatory effects.

Specific examples of potentially life-threatening acute fluid overload affecting the primary organ systems of the body include Brain—cognitive impairment, Heart—edema, arrythmia, congestive heart failure, Lungs—pulmonary edema, pleural efflusion, hypoxemia, hypoxia, Kidney—reduced glomerular filtration rate, acute injury, proteinuria, Liver—impaired liver functions including albuminemia.

Hence, it is important to avoid the major consequences which happen by administration error or particularly if pegylated blood product is infused in inadequate amount, inadequate flow rate and inadequate time period The present invention discloses an improved infusion process by infusing an adequate amt of PEGylated hemoglobin into the subject in need thereof in at least two cycles. The present invention also maintains adequate flow rate and blood volume to avoid any side effects.

### SUMMARY OF THE INVENTION

Disclosed herein, in some embodiments, is a process of infusing PEGylated HB-CO (PEG-HB-CO) in a subject in need thereof, the process comprising:
a. infusing first cycle of PEGylated HB-CO (PEG-HB-CO) in the subject; and
b. infusing second cycle of PEGylated HB-CO (PEG-HB-CO in the subject;
wherein the subject is intravenously and/or subcutaneously infused with PEG-Hb-CO in at least two cycles;
wherein the infusion process is safe to subject; wherein the infusion process improves one or more conditions selected from tissue oxygenation, maintains blood pressure, and controls adverse effect of hypervolemia in the subject.

Disclosed herein, in some embodiments, is a process for improved infusion of PEGylated HB-CO (PEG-HB-CO) in a subject in need thereof, the process comprising:
a. infusing first cycle of PEGylated HB-CO (PEG-HB-CO) in the subject; and
b. infusing second cycle of PEGylated HB-CO (PEG-HB-CO in the subject;
wherein the subject is intravenously and/or subcutaneously infused with PEG-Hb-CO in at least two cycles;
wherein the infusion process is safe to subject; wherein the infusion process improves one or more conditions selected from tissue oxygenation, maintains blood pressure, and controls adverse effect of hypervolemia in the subject.

In some embodiments, the subject safety is improved. In some embodiments, there is no toxicity observed in the subject. In some embodiments, there is no death observed in the subject.

In some embodiments, the subject safety is improved and there is no toxicity observed in the subject.

In some embodiments, the subject safety is improved and there is no death observed in the subject.

In some embodiments, the subject is a patient.

Disclosed herein, in some embodiments, is a process of infusing PEGylated HB-CO (PEG-HB-CO) in the subject in need thereof, the process comprising:
a. infusing first cycle of PEGylated HB-CO (PEG-HB-CO) in the subject at a suitable flow rate; and
b. infusing second cycle of PEGylated HB-CO (PEG-HB-CO) in the subject at a suitable flow rate;
wherein the PEG-HB-CO is administered in at least 2 cycles,
wherein the first cycle of PEG-HB-CO has a higher flow rate than the second cycle, and
wherein, the first cycle delivers a lower amount of PEG-HB-CO than the second cycle.

Disclosed herein, in some embodiments, is a process for improved infusion of PEGylated HB-CO (PEG-HB-CO) in the subject in need thereof, the process comprising:
a. infusing first cycle of PEGylated HB-CO (PEG-HB-CO) in the subject at a suitable flow rate; and
b. infusing second cycle of PEGylated HB-CO (PEG-HB-CO) in the subject at a suitable flow rate;
wherein the PEG-HB-CO is administered in at least 2 cycles,
wherein the first cycle of PEG-HB-CO has a higher flow rate than the second cycle, and
wherein, the first cycle delivers a lower amount of PEG-HB-CO than the second cycle.

In some embodiments, the subject is not able to maintain tissue oxygenation because of obstruction in a blood vessel.

In some embodiments, the subject safety is improved.

Disclosed herein, in some embodiments, is a process for infusing PEGylated HB-CO (PEG-HB-CO) in the subject, wherein subject safety is improved by improving one of more conditions selected from tissue oxygenation, maintains blood pressure, and controls adverse effect of hypervolemia in the subject.

Disclosed herein, in some embodiments, is a process for improved infusion of PEGylated HB-CO (PEG-HB-CO) in the subject, wherein patient safety is improved by improving one of more conditions selected from tissue oxygenation, maintains blood pressure, and controls adverse effect of hypervolemia in the subject.

Disclosed herein, in some embodiments, is a process of infusing PEGylated HB-CO (PEG-HB-CO) in the subject, wherein the infusion process controls adverse effects of hypervolemia.

Disclosed herein, in some embodiments, is a process for improved infusion of PEGylated HB-CO (PEG-HB-CO) in the subject, wherein the infusion process controls adverse effects of hypervolemia.

Disclosed herein, in some embodiments, is a process of infusing PEGylated HB-CO (PEG-HB-CO) in the subject, wherein the infusion process maintains the blood pressure of the subject.

Disclosed herein, in some embodiments, is a process for improved infusion of PEGylated HB-CO (PEG-HB-CO) in the subject, wherein the infusion process maintains the blood pressure of the subject.

Disclosed herein, in some embodiments, is a process of infusing PEGylated HB-CO (PEG-HB-CO) in the subject, wherein the blood pressure is maintained in clinically acceptable range.

Disclosed herein, in some embodiments, is a process for improved infusion of PEGylated HB-CO (PEG-HB-CO) in the subject, wherein the blood pressure is maintained in clinically acceptable range.

Disclosed herein, in some embodiments, is a process of infusing PEGylated HB-CO (PEG-HB-CO), wherein the process maintains the tissue oxygenation in the subject.

Disclosed herein, in some embodiments, is a process for improved infusion of PEGylated HB-CO (PEG-HB-CO), wherein the process maintains the tissue oxygenation in the subject.

Disclosed herein, in some embodiments, is a process of infusing PEGylated HB-CO (PEG-HB-CO), wherein the process improves subject safety.

Disclosed herein, in some embodiments, is a process for improved infusion of PEGylated HB-CO (PEG-HB-CO), wherein the process improves subject safety.

Disclosed herein, in some embodiments, is a process of infusing PEGylated HB-CO (PEG-HB-CO), wherein the process controls adverse effect of hypervolemia.

Disclosed herein, in some embodiments, is a process for improved infusion of PEGylated HB-CO (PEG-HB-CO), wherein the process controls adverse effect of hypervolemia.

Disclosed herein, in some embodiments, is a process of infusing PEGylated HB-CO (PEG-HB-CO), wherein the administration of PEG-HB-CO is safe to subject.

Disclosed herein, in some embodiments, is a process for improved infusion of PEGylated HB-CO (PEG-HB-CO), wherein the administration of PEG-HB-CO is safe to subject.

In some embodiments, the subject is a patient.

Disclosed herein, in some embodiments, is a process of reducing the incidence and/or alleviating one or more symptoms characterized by the lack of blood supply in a subject, the process comprising:
a. infusing first cycle of PEGylated HB-CO (PEG-HB-CO) in the subject; and
b. infusing second cycle of PEGylated HB-CO (PEG-HB-CO) in the subject;
wherein, the subject is administered with a therapeutically effective amount of PEG-HB-CO in at least two cycles.

Disclosed herein, in some embodiments, is a process of infusing PEGylated-HB-CO (PEG-HB-CO) in a subject in need thereof, the process comprising:
a. infusing first cycle of PEGylated HB-CO (PEG-HB-CO) in the subject; and
b. infusing second cycle of PEGylated HB-CO (PEG-HB-CO) in the subject;
wherein, the subject is intravenously and/or subcutaneously infused with PEG-Hb-CO in at least two cycles.

Disclosed herein, in some embodiments, is an improved infusion process of PEGylated-HB-CO (PEG-HB-CO) in a subject in need thereof, the process comprising:
a. infusing first cycle of PEGylated HB-CO (PEG-HB-CO) in the subject; and
b. infusing second cycle of PEGylated HB-CO (PEG-HB-CO) in the subject;
wherein, the subject is intravenously and/or subcutaneously infused with PEG-Hb-CO in at least two cycles.

In some embodiments, the present invention discloses an improved infusion process of PEGylated HB-CO in the subject in need thereof comprising:
a. infusing PEGylated HB-CO in suitable amount and at suitable flow rate in first cycle;
b. infusing PEGylated HB-CO in suitable amount and at suitable flow rate in second cycle; wherein the first cycle has higher flow rate than the second cycle.

In some embodiments, the first cycle has higher flow rate at least about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, and about 100% than the second cycle.

In some embodiments, the first cycle has higher flow rate at least about 20% to about 80% higher than the second cycle.

In some embodiments, the first cycle has higher flow rate at least about 40% to about 60% higher than the second cycle.

In some embodiments, the first cycle has higher flow rate at least about 40% to about 50% higher than the second cycle.

Disclosed herein, in some embodiments, is a process of infusing PEGylated hemoglobin in a subject in the need thereof comprising,
a. infusing PEGylated HB-CO in suitable amount and at suitable flow rate in first cycle; and
b. infusing PEGylated HB-CO in suitable amount and at suitable flow rate in second cycle,
wherein the first cycle delivers lower amount of infused PEGylated hemoglobin than the second cycle.

Disclosed herein, in some embodiments, is an improved infusion process of PEGylated hemoglobin in a subject in the need thereof comprising,
a. infusing PEGylated HB-CO in suitable amount and at suitable flow rate in first cycle; and
b. infusing PEGylated HB-CO in suitable amount and at suitable flow rate in second cycle,
wherein the first cycle delivers lower amount of infused PEGylated hemoglobin than the second cycle.

In some embodiments, the first cycle delivers lower amount of pegylated hemoglobin at least by about 5%, about 10%, about 15%, about 20%, about 25%, about 30%, about 35%, about 40%, about 45%, about 50%, about 55%, about 60%, about 65%, and about 70% than second or subsequent cycle.

In some embodiments, the first cycle delivers a lower amount of pegylated hemoglobin at least about 5%, to about 70% than the second or subsequent cycle.

In some embodiments, the first cycle delivers a lower amount of pegylated hemoglobin at least about 20%, to about 70% than the second or subsequent cycle.

In some embodiments, the first cycle delivers a lower amount of pegylated hemoglobin at least about 50%, to about 70% than the second or subsequent cycle.

In some embodiments, the first cycle delivers a lower amount of pegylated hemoglobin at least about 60%, to about 70% than the second or subsequent cycle.

In some embodiments, the first cycle administers about 40% of the total dose and wherein the second cycle administers about 60% of the total dose.

Disclosed herein, in some embodiments, is a process of administering PEGylated HB-CO (PEG-HB-CO) to a subject in need thereof to treat at least one symptom characterized by the lack of blood supply in the subject, the process comprising:
a. infusing therapeutically effective amount of PEG-Hb-CO in the subject in first cycle; and
b. infusing therapeutically effective amount of PEG-Hb-CO in the subject in second cycle;
wherein, the subject is intravenously and/or subcutaneously infused with PEG-Hb-CO in at least two cycles;
wherein, the first cycle has a flow rate about 5% to about 100% higher than the second cycle, or about 20% to 80% higher than the second cycle;
wherein, the first cycle delivers about 5% to about 70% or about 20% to about 70% or about 50% to about 70% lower amount of infused PEG-Hb-CO than second cycle;
wherein, the process of PEGylated HB-CO (PEG-HB-CO) infusion does not lead to a transient increase in the blood pressure with such a level which is clinically unacceptable.

Disclosed herein, in some embodiments, is an improved infusion process of administering PEGylated HB-CO (PEG-HB-CO) in a subject in need thereof to treat at least one symptom characterized by the lack of blood supply in the subject, the process comprising:
a. infusing therapeutically effective amount of PEG-Hb-CO in the subject in first cycle; and
b. infusing therapeutically effective amount of PEG-Hb-CO in the subject in second cycle;
wherein, the subject is intravenously and/or subcutaneously infused with PEG-Hb-CO in at least two cycles;
wherein, the first cycle has a flow rate about 5% to about 100% higher than the second cycle, or about 20% to 80% higher than the second cycle;
wherein, the first cycle delivers about 5% to about 70% or about 20% to about 70% or about 50% to about 70% lower amount of infused PEG-Hb-CO than second cycle;
wherein, the process of PEGylated HB-CO (PEG-HB-CO) infusion does not lead to a transient increase in the blood pressure with such a level which is clinically unacceptable.

In some embodiments, the subject is administered with therapeutically effective amount of PEG-HB-CO for about 2 hours to about 8 hours in at least 2 cycles.

In some embodiments, the subject is administered with therapeutically effective amount of PEG-HB-CO for about 2 hours in at least 2 cycles.

In some embodiments, the first cycle delivers PEG-HB-CO in the range of 5 ml/kg to about 225 ml/kg wherein the total blood volume is at least 500 ml.

In some embodiments, the first cycle delivers PEG-HB-CO in the range of 100 ml/kg to about 200 ml/kg wherein the total blood volume is at least 500 ml.

In some embodiments, the first cycle lasts for at least 5 minutes, about 10 minutes, about15 minutes, about 20 minutes, about 25 minutes, about 30 minutes, and about 35 minutes.

In some embodiments, the first cycle lasts for at least about 15 minutes, about 20 minutes, about 25 minutes, about 30 minutes, and about 35 minutes.

In some embodiments, the flow rate maintained during first cycle is at least by 3 ml/min, about 4 ml/min, about 5 ml/min, about 6 ml/min, about 6.5 ml/min, about 7 ml/min, 8 ml/min based on the weight of subject and effective amount of pegylated hemoglobin needs to be administrated.

In some embodiments, the flow rate maintained during first cycle is about 4 ml/min, about 5 ml/min, about 6 ml/min, about 6.5 ml/min, about 7 ml/min based on the weight of subject and effective amount of pegylated hemoglobin needs to be administrated.

In some embodiments, the second cycle delivers higher amount of PEGylated HB-CO in second cycle at least about 275 ml/kg, about 300 ml/kg, about 350 ml/kg, about 400 ml/kg, about 450 ml/kg, and bout 500 ml/kg wherein the total blood volume is at least 500 ml.

In yet another embodiment, the second cycle delivers higher amount of PEGylated HB-CO in second cycle at least about 300 ml/kg, about 350 ml/kg, about 400 ml/kg wherein the total blood volume is at least 500 ml

In some embodiments, the flow rate maintained during second cycle is about 10 min, about 20 min, about 30 min, about 40 min, about 50 min, about 60 min, about 70 min, about 80 min, about 90 min and about 100 min.

In some embodiments, the flow rate maintained during second cycle is about 30 min, about 40 min, about 50 min, about 60 min, about 70 min, about 80 min, about 90 min.

In some embodiments, the flow rate is maintained during second cycle is at least by 1 ml/min, about 2 ml/min, about 2.5 ml/min, about 3 ml/min, about 3.3 ml/min, about 4 ml/min, about 4.5 ml/min based on the weight of subject and effective amount of pegylated hemoglobin needs to be administrated.

In some embodiments, the flow rate maintained in second cycle will always be lower than flow rate maintained in first cycle.

Disclosed herein, in some embodiments, is a process of infusing PEGylated HB-CO (PEG-HB-CO) in a subject in the need thereof to reduce the incidence and/or alleviate at least one of the adverse effects selected from the following:
a. transient increase in the blood pressure with such a level which is clinically unacceptable;
b. cardiovascular events;
c. haemorrhagic shock;
d. intracranial haemorrhage;
e. hypoxia;
f. necrosis;
g. myocardial injury;
h. acute myocardial infarction;
i. organ injury such as brain or kidney injury.
j. heart rate, respirations, and temperature;
k. hypertension;
l. pulmonary hypertension; and
m. ischemic stroke,
wherein, the subject is intravenously and/or subcutaneously infused with PEG-HB-CO in at least two cycles;
wherein, the flow rate in the first cycle is about 5% to about 100% or about 20% to about 80% or about 40% to about 60% higher than the second cycle;
wherein, the first cycle delivers a lower amount of pegylated hemoglobin at least about 5%, to about 70% than the second or subsequent cycle.

Disclosed herein, in some embodiments, is an improved infusion process of PEGylated HB-CO (PEG-HB-CO) in a subject in the need thereof to reduce the incidence and/or alleviate at least one of the adverse effects selected from the following:
a. transient increase in the blood pressure with such a level which is clinically unacceptable;
b. cardiovascular events;
c. haemorrhagic shock;
d. intracranial haemorrhage;
e. hypoxia;
f. necrosis;
g. myocardial injury;
h. acute myocardial infarction;
i. organ injury such as brain or kidney injury.
j. heart rate, respirations, and temperature;
k. hypertension;
l. pulmonary hypertension; and
m. ischemic stroke,
wherein, the subject is intravenously and/or subcutaneously infused with PEG-HB-CO in at least two cycles;
wherein, the flow rate in the first cycle is about 5% to about 100% or about 20% to about 80% or about 40% to about 60% higher than the second cycle;
wherein, the first cycle delivers a lower amount of pegylated hemoglobin at least about 5%, to about 70% than the second or subsequent cycle.

Disclosed herein, in some embodiments, is a process of administering PEGylated HB-CO (PEG-HB-CO) to a subject in need thereof to treat at least one symptom characterized by the lack of blood supply in the subject, the process comprising:
a. infusing therapeutically effective amount of PEG-Hb-CO in the subject in first cycle; and
b. infusing therapeutically effective amount of PEG-Hb-CO in the subject in second cycle;
wherein, the subject is intravenously and/or subcutaneously infused with PEG-Hb-CO in at least two cycles;
wherein, the first cycle has a flow rate about 5% to about 100% higher than the second cycle, or about 20% to 80% higher than the second cycle;
wherein, the first cycle delivers about 5% to about 70% or about 20% to about 70% or about 50% to about 70% lower amount of infused PEG-Hb-CO than second cycle;
wherein, the process of PEGylated HB-CO (PEG-HB-CO) infusion does not lead to a transient increase in the blood pressure with such a level which is clinically unacceptable.

Disclosed herein, in some embodiments, is an improved infusion process of administering PEGylated HB-CO (PEG-HB-CO) in a subject in need thereof to treat at least one symptom characterized by the lack of blood supply in the subject, the process comprising:
a. infusing therapeutically effective amount of PEG-Hb-CO in the subject in first cycle; and
b. infusing therapeutically effective amount of PEG-Hb-CO in the subject in second cycle;
wherein, the subject is intravenously and/or subcutaneously infused with PEG-Hb-CO in at least two cycles;
wherein, the first cycle has a flow rate about 5% to about 100% higher than the second cycle, or about 20% to 80% higher than the second cycle;
wherein, the first cycle delivers about 5% to about 70% or about 20% to about 70% or about 50% to about 70% lower amount of infused PEG-Hb-CO than second cycle;
wherein, the process of PEGylated HB-CO (PEG-HB-CO) infusion does not lead to a transient increase in the blood pressure with such a level which is clinically unacceptable.

Disclosed herein, in some embodiments, is a method for treating a subject suffering from inadequate supply of blood to a tissue or organs, wherein the method comprises:
a. administering the PEGylated HB-CO (PEG-HB-CO) in the subject through infusion; and
b. improving the subject condition,
wherein the infusion is performed in at least two cycles, and
wherein, the subject is administered with a therapeutically effective amount of PEG-HB-CO in at least two cycles.

Disclosed herein, in some embodiments, is a method for administering a PEGylated HB-CO (PEG-HB-CO) to a subject, wherein the method comprises:
a. infusing therapeutically effective amount of PEG-Hb-CO in the subject in first cycle; and
b. infusing therapeutically effective amount of PEG-Hb-CO in the subject in second cycle,
wherein, the subject is intravenously and/or subcutaneously infused with PEG-Hb-CO in at least two cycles;
wherein the administration is safe to subject; wherein the administration is useful in the treatment of a diseases condition.

In some embodiments, the process or method maintains the blood pressure in the subject in clinically acceptable range.

In some embodiments, the process or method exhibits improvement by maintaining systolic pressure in the clinically acceptable range between the range of 120 mm Hg to about 180 mm Hg.

In yet another embodiment, the process or method exhibits improvement by maintaining diastolic pressure in the clinically acceptable range below 95 mm Hg.

Disclosed herein, in some embodiments, is a process of administering PEGylated HB-CO (PEG-HB-CO) to a subject in need thereof to treat at least one symptom characterized by the lack of blood supply in the subject, the process comprising:
a. infusing first cycle of PEGylated HB-CO (PEG-HB-CO) in the subject; and
b. infusing second cycle of PEGylated HB-CO (PEG-HB-CO) in the subject;
wherein the process exhibits improvement by obtaining systolic pressure in the clinically acceptable range between the range of 120 mm Hg to about 180 mm Hg, and
wherein the process exhibits improvement by obtaining diastolic pressure below 95 mm Hg.

Disclosed herein, in some embodiments, is an improved infusion process of administering PEGylated HB-CO (PEG-HB-CO) to a subject in need thereof to treat at least one symptom characterized by the lack of blood supply in the subject, the process comprising:
a. infusing first cycle of PEGylated HB-CO (PEG-HB-CO) in the subject; and
b. infusing second cycle of PEGylated HB-CO (PEG-HB-CO) in the subject;
wherein the process exhibits improvement by obtaining systolic pressure in the clinically acceptable range between the range of 120 mm Hg to about 180 mm Hg, and
wherein the process exhibits improvement by obtaining diastolic pressure below 95 mm Hg.

In some embodiments, the process or method reduces the stroke risk measured by National Institutes of Health Stroke Scale (NIHSS) score.

In some embodiments, the process or method exhibits improvement by reducing National Institutes of Health Stroke Scale (NIHSS) score.

In some embodiments, the process or method exhibits improvement by obtaining the stroke risk between about 0 to about 16 NIHSS score.

In some embodiments, the subject NIHSS score is analysed on day 0, day 1, or day 90, or any combination thereof. In some embodiments, the subject NIHSS score is analysed on day 0. In some embodiments, the subject NIHSS score is analysed on day 1. In some embodiments, the subject NIHSS score is analysed on day 90.

In some embodiments, the subject NIHSS score is analysed on day 0 or 1 on day 90.

In some embodiments, the subject mRS score is analysed on day 0, day 1, or day 90, or any combination thereof. In some embodiments, the subject mRS score is analysed on day 0. In some embodiments, the subject mRS score is analysed on day 1. In some embodiments, the subject mRS score is analysed on day 90.

In some embodiments, the subject mRS score is analysed on day 0 or 1 on day 90.

In some embodiments, the process or method reduces the risk of disability in the subject measured by Modified Rankin Scale (mRS).

In some embodiments, the process or method exhibits improvement by obtaining the disability risk between about 0 to about 3 mRS score.

In some embodiments, the rate and volume of PEG-Hb-CO are monitored by a physiological measurement. In some embodiments, the physiological measure is an acute measurement. In some embodiments, the acute measurement is blood pressure, arterial blood gases, heart rate, respiration rate, or conscious activity among others.

In some embodiments, the rate and volume of PEG-Hb-CO are instead monitored by a physiological measurement including but not limited to acute measures such as blood pressure, arterial blood gases, heart rate, respiration rate, and conscious activity among others.

In some embodiments, the intravenous infusion of PEGylated hemoglobin can be administrated to a subject before standard of care. For example, a trauma subject may be administered PEG-Hb-CO to increase their blood pressure, then receiving standard of care vasopressors for refractory hypotension stabilizing subject blood pressure. The second and/or continued administration may allow for lower vasopressor use that may also allow for vasopressor tapering thereby limiting risks of advanced peripheral limb ischemia and amputation.

In some embodiments, the intravenous infusion of PEGylated hemoglobin can be administrated to a subject before the administration of intravenous thrombolytica including recombinant tissue plasminogen activator (rtPA), or mechanical thrombectomy, or both in either order.

In some embodiments, the intravenous infusion of PEGylated hemoglobin sufficient enough to deliver enough oxygen to target tissues which is ischemic or suffering from ischemia. This may include limb ischemia/tissue hypoxia due to poor circulation or vessel blockage. This could be due to anemia wherein the drug increases blood oxygen carrying capacity, and/or improvements to the microcirculation thereby improving normal physiological parameters. This would reduce risks of poor outcomes with acute stroke, myocardial infarction, acute chest syndrome, kidney and liver injury and loss of function.

In some embodiments, the subject is selected from animal, mammals, or human subject.

In some embodiments, the subject is human.

In some embodiments, the subject is a patient.

In some embodiments, the PEG-HB-CO is infused or administered in a dose-dependent manner. In some embodiments, the PEG-HB-CO is infused or administered in a dose-dependent manner, wherein PEG-HB-CO dose is dependent on the subject body weight.

In some embodiments, the PEG-HB-CO dose in the dose dependent manner is selected from about 300 mg/kg to about 20,000 mg/kg.

In some embodiments, wherein the subject body weight is 62.5 and above; wherein the dose is administrated to the subject is about 20,000 mg/kg.

In some embodiments, the first cycle administrated 8000 mg of the dose and wherein the second cycle administrated 1200 mg of the dose.

In some embodiments, the invention provides a method for reducing incidence of or treating at least one symptom in an individual, comprising administering to the individual an effective amount of pegylated hemoglobin wherein said pegylated hemoglobin is perfused in individual in at least two cycles.

Disclosed herein is an improved infusion process of PEG-HB-CO in the subject in need to treat at least one symptom characterized by the lack of blood supply in the subject, wherein the effects of PEG-HB-CO (PP-007) on Sickle Cell Disease in combination with standard of care treatment were analysed. PEG-HB-CO was administered in at least two cycles in the subject, wherein the flow rate of the first cycle is higher than the second cycle, and the first cycle delivers a lower amount of PEG-HB-CO to the subject. The flow rate of the first cycle is 6.67 ml/min, and the dose volume is 200 ml/kg, wherein the infusion rate of the second cycle is 3.33 ml/min and the infusion dose volume is 300 ml/kg. It was observed when PEG-HB-CO is administered in a subject in at least two cycles, the blood pressure of the SCD patient is maintained in the clinically acceptable range. Additionally, the adverse effect of hypervolemia was controlled, thereby controlling hypoxia and improving tissue oxygenation rate.

The present invention enables the use of two cycle administration of PEG-HB-CO in the subject in need thereof suffering ischemic stroke where the subject is found stable and is able to maintain the blood pressure in clinically acceptable range and has not shown any symptom of hypervolemia.

The 2 cycle infusion process disclosed herein is suitable for the treatment of disease where lack of oxygenation of hypoxia or lack of blood supply is a key concern. For example, the 2 cycle infusion process disclosed herein is suitable for treatment of a disease selected from the group consisting of: ischemic stroke, Sickle Cell disease, anemia, cardiovascular events, haemorrhagic shock, intracranial haemorrhage, hypoxia, necrosis, myocardial injury, acute myocardial infarction, organ injury, brain injury, kidney injury, heart rate, respirations, and temperature, hypertension, and pulmonary hypertension.

### DETAILED DESCRIPTION OF THE INVENTION

The terms “pegylated hemoglobin”, “pegylated blood”, “blood substitute,” “resuscitation fluid”, “PEG-Hb”, “PEG-CO-Hb”, “-based oxygen carrier”, “HBOC”, “PEGylated HB-CO”, “PP-007”, “PEG-HB-CO”, “PEGylated bovine hemoglobin”, “PEGylated carboxyhemoglobin”, and “PEG-Hb/HS” are interchangeable and refer to the PEGylated Hb compositions of the invention and formulations incorporating these compositions. The terms also carry with them the disclosure of an exemplary use of the composition and its formulation. For example, a “blood substitute” is of use to replace blood in the context of, e.g., trauma, stroke, ischemia/reperfusion injury, surgery, anemia or other injuries, insults and diseases in which a blood transfusion might be indicated. These terms, as used herein, also refer to Hb formulations capable of delivering oxygen or carbon monoxide to a tissue. These formulations are of use in injuries, insults and diseases characterized by the subject having adequate blood volume, yet the blood has inadequate ability to carry and/or deliver oxygen or carbon monoxide to tissues. The PEG-derivatives are formulated in hypotonic, isotonic or hypertonic salt solutions. Thus, exemplary deoxygenated PEG-Hb composition in which the Fe (II) is unbound or is bound to CO can be formulated in isotonic or hypertonic solution. Similarly, exemplary oxygenated PEG-Hb can be formulated in isotonic or hypertonic carriers. As used herein the term “pegylated hemoglobin” or “pegylated blood” or “PEG-Hb” or “PEGylated HB-CO” or “PP-007” or “PEG-HB-CO” or “PEGylated bovine hemoglobin” or “PEGylated carboxyhemoglobin” which are interchangeable refers to pegylated oxygenated hemoglobin or carbon monoxide associated pegylated hemoglobin.

“Hemoglobin” as used herein refers to an oxygen-binding (or CO-binding), active polypeptide that is not chemically cross-linked through treatment with chemical cross-linking agents, e.g., dialdehydes, etc. An exemplary is the native protein with no modifications other than the conjugation of one or more PEG (e.g., m-PEG) moieties. As used herein, “substantially free of chemical cross-linking agents,” refers to molecules that are not purposely cross-linked with chemical cross-linking agents. These preparations include less than 5%, less than 3% or less than 1% cross-linked.

The term “CO” refers to carbon monoxide.

In some embodiments, the intravenous infusion of PEGylated-HB-CO is administered in subject before standard of care.

In healthcare, “standard of care” refers to the accepted level of medical treatment that a reasonably competent and qualified healthcare provider would provide under similar circumstances. It's a benchmark used to determine if professional obligations to patients have been met and is relevant in medical malpractice lawsuits.

In some embodiments, the standard of care for acute ischemic stroke involves rapid assessment, stabilization, and potentially thrombolytic therapy (IV alteplase) within 4.5 hours of symptom onset, along with consideration for endovascular therapy (mechanical thrombectomy) in selected cases.

“Ischemic stroke” is the most common type of stroke, occurs when a blood clot blocks or plugs an artery, leading to reduced blood flow to the brain. Clinical endpoints used to measure the impact of ischemic stroke interventions primarily focus on functional outcomes, such as disability and recovery, as well as assessing the severity and extent of stroke. The clinical endpoints are Modified Rankin Scale (mRS), National Institutes of Health Stroke Scale (NIHSS), and Barthel Index.

“Sickle cell disease” or “SCD” is a genetic disorder where abnormal hemoglobin causes red blood cells to become rigid and crescent-shaped, hindering their ability to carry oxygen and leading to blockages in blood vessels. Clinical endpoints in measuring SCD primarily focus on acute pain crises (vaso-occlusive crises or VOCs) and assessing the impact of treatments on these crises and related complications. The clinical endpoints are Reduction in VOC Frequency, Severity of Pain, End-organ damage

“Anemia” is a condition where the number of red blood cells or the hemoglobin concentration within them is lower than normal. This reduces the blood's capacity to carry oxygen, leading to symptoms like fatigue and shortness of breath. Clinical endpoints for measuring anemia include improvements in hemoglobin levels, hematocrit, and overall quality of life.

The term “National Institutes of Health Stroke Scale or “NIHSS” is a standardized tool used to assess the severity of stroke, particularly ischemic stroke, and predict subject outcomes, helping healthcare providers evaluate and monitor subject. The National Institutes of Health Stroke Scale (NIHSS) scores range from 0 to 42, with higher scores indicating more severe stroke-related neurological deficits. Herein NIHSS score of 0 indicates normal neurological function and no evidence of stroke-related deficits score 1 to score 4 indicates a minor stroke, score 5-15 indicates a moderate stroke severity, score 15-20 indicates a moderate to severe stroke and score 21-42 indicates severe stroke in subject.

The term “Modified Rankin Scale” or “mRS” are interchangeable and refer to a widely used ordinal scale to assess functional outcomes after stroke, ranging from 0 (no symptoms) to 6 (death), with higher scores indicating greater disability. It's often used in clinical trials and clinical practice to evaluate stroke recovery and is typically measured at 90 days post-stroke. When Modified rankin scale score of 0 in the context of ischemic stroke indicates no residual symptoms at all, representing a fully independent and healthy state following the stroke, mRS score of 1 indicates no significant disability, despite symptoms, meaning the subject is able to carry out all usual duties and activities, mRS score of 2 indicates a slight disability, where the subject is unable to perform all previous activities but can still look after their own affairs without assistance, mRS score of 3 in the context of ischemic stroke indicates moderate disability, requiring some help but still able to walk without assistance, mRS score of 4 in the context of ischemic stroke indicates moderately severe disability, meaning the subject is unable to walk without assistance and unable to attend to their own bodily needs without help, mRS score of 5 in the context of ischemic stroke indicates severe disability, requiring constant nursing care and attention, with the subject being bedridden and incontinent, mRS score of 6 signifies that the subject has died due to disability.

In some embodiments, the present invention discloses an improved infusion process of PEG HB-CO, wherein PEG-HB-CO is administered to a subject before administering the subject with Intravenous (IV) thrombolytic therapy including recombinant tissue plasminogen activator (rt-PA), or mechanical thrombectomy, or both in any order.

In some embodiments, the standard of care for anemia focuses on identifying and addressing the underlying cause, with treatment ranging from dietary changes and supplements to blood transfusions or bone marrow transplants, depending on the type and severity of anemia.

In another embodiment, the standard of care for hypoxia involves ensuring a patent airway, administering supplemental oxygen, and addressing the underlying cause, potentially including mechanical ventilation or other supportive measures. The term “Clinically acceptable range” for the disease conditions here is the range of the blood pressure that is maintained after the administration of PP-007. Herein the systolic pressure is maintained in the clinically accepted range of 120 mm/Hg to 180 mm/Hg. And the diastolic pressure in a subject is maintained below 95 mm/Hg after the administration of PP-007.

The term “peptide conjugate,” and “conjugate” refer to species of the invention in which a polypeptide is conjugated with a water-soluble polymer, e.g., poly(ethylene glycol) (PEG), as set forth herein.

“Deoxygenated hemoglobin” refers to hemoglobin in which the Fe (II) atom is bound to a species other than oxygen (e.g., carbon monoxide) or is not bound to oxygen or any other species.

The term “therapy” refers to “treating” or “treatment” of a disease or condition including providing relief from the symptoms or side-effects of the disease or obtaining beneficial or desired clinical results. For purposes of this invention, beneficial or desired clinical results include, but are not limited to, one or more of the following: improvement in the treatment of a diseases or chronic diseases selected from hemorrhagic shock, hypovolemia, alloimmunization, sickle cell anaemia, beta thalassemia, chemotherapy toxicity, red blood cell aplasia, myocardial infarction, hypoxic ischemic encephalopathy, ischemic stroke, ischemia/reperfusion injury, trauma, hypoxia, a and the like limb ischemia, CO poisoning, venom/toxin, environmental poisoning, tumor hypoxia, anemia including lessening severity, alleviation of other blood infusion associated symptoms, reducing frequency of recurrence of symptoms or adverse events associated with blood infusion.

The term “effective amount” or “therapeutically effective amount” refers to “effective dosage” or “adequate amount” of blood or pharmaceutical composition thereof in an amount sufficient to effect beneficial or desired results. For therapeutic use, beneficial or desired results include clinical results such as reducing the adverse events associated with diseases or chronic diseases selected from haemorrhagic shock, stroke, ischemia/reperfusion injury, trauma, hypoxia and the like and increasing the quality of life of those suffering from the disease, decreasing the dose of other medications required to treat the disease, enhancing effect of another medication, and/or delaying the progression of the disease of patients. In exemplary embodiments, this term refers to any amount of a conjugate of the invention (or a formulation including a conjugate of the invention) sufficient to repay at least 50%, at least 60%, at least 70%, at least 80%, at least 90% or up to about 100% of tissue or organ oxygen debt attributable to disease, insult or injury. When used in the context of delivery of CO to a tissue, this term refers to an amount administered sufficient to derive a detectable therapeutic effect from the delivery of CO to a tissue. An effective dosage can be administered in at least two cycles of administrations. In some embodiments, an effective dosage can be administrated in at least three cycle or four cycle.

In some embodiments, the therapeutic effective amount of PEG-HB-CO is selected from about 300 mg/kg to about 20,000 mg/kg. In another embodiment, the therapeutic effective amount of PEG-HB-CO is selected from about 1000 mg/kg to about 20,000 mg/kg.

The term “first dose” refers to intravenous infusion of PEGylated hemoglobin or PEGylated hemoglobin containing carbon monoxide to a subject administrate in a first cycle.

The term “first cycle” refers to the administration of first dose of PEG-Hb or PEGylated hemoglobin by intravenous infusion in effective amount at adequate flow rate for the treatment of diseases and prevention, amelioration, or treatment of any side effect associated with improper perfusion of said pegylated Hb. First cycle maintains higher flow rate than second or subsequent cycle at least by about 5%, about 10%, about 15%, about 20%, about 25%, about 30%, about 35%, about 40%, about 45%, about 50%, about 55%, about 60%, about 65%, about 70%, about 75%, about 80%, about 90%, about 95% and about 100% than the second cycle.

In some embodiments, the first cycle has lower amount of pegylated hemoglobin in first cycle at least about 100 ml/kg, about 150 ml/kg, about 200 ml/kg, about 225 ml/kg wherein the total blood volume is at least 500 ml.

In some embodiments, the first cycle last for least 5 min, about 10 min, about15 min, about 20 min, about 25 min, about 30 min, about 35 min.

In some embodiments, the flow rate is maintained during first cycle is at least by 3 ml/min, about 4 ml/min, about 5 ml/min, about 6 ml/min, about 6.5 ml/min, about 7 ml/min, 8 ml/min based on the weight of subject and effective amount of pegylated hemoglobin needs to be administrated.

The term “second dose” refers to intravenous infusion of PEG-Hb or PEGylated hemoglobin to a subject administrates in a second cycle.

The term “second cycle” refers to the administration of PEG-Hb or PEGylated hemoglobin by intravenous infusion with low flow rate or infusion rate and has higher amount of pegylated hemoglobin in second cycle than first cycle. In some embodiments the second cycle last for least 5 min, about 10 min, about15 min, about 20 min, about 25 min, about 30 min, about 35 min.

In some embodiments, the flow rate is maintained during second cycle is at least by 1 ml/min, about 2 ml/min, about 2.5 ml/min, about 3 ml/min, about 3.3 ml/min, about 4 ml/min, about 4.5 ml/min based on the weight of subject and effective amount of pegylated hemoglobin needs to be administrated.

The term “at least two cycles” refers to intravenous infusion of PEGylated hemoglobin by first cycle or second cycle and can be optionally increase to subsequent third cycle and forth cycle.

The term “subsequent dose” refers to intravenous infusion of PEG-Hb or PEGylated hemoglobin to a subject after the completion of second dose and second cycle. It can be administrated as third or fourth dose based the subject need.

The term “subsequent cycle” refers to intravenous infusion of PEG-Hb or PEGylated hemoglobin to a subject after the completion of second cycle. It includes third or fourth dose based the subject need. In some embodiments the subsequent cycle comprises the same dose and/or flow rate identical to second cycle.

The term “Adverse events” refers to AE is defined as any untoward medical occurrence in a clinical investigation subject administered a pharmaceutical product, which does not necessarily have a causal relationship with the treatment. An AE can therefore be any unfavourable and/or unintended sign (including an abnormal laboratory finding), symptom, or disease temporally associated with the use of an investigational product, whether or not related to the investigational product.

The term “myocardial infarction” refers to cardiac vessel ischemia and blockage resulting in cardiomyocyte cell death. Progressive and potentially when the lung blood pressure is higher than the cardiac blood pressure thereby preventing proper blood oxygenation.

The term “ameliorating” or “ameliorate” refers to any indicia of success in the treatment of a pathology or condition, including any objective or subjective parameter such as abatement, remission or diminishing of symptoms or an improvement in a subject's physical or mental well-being. Amelioration of symptoms can be based on objective or subjective parameters; including the results of a physical examination and/or a psychiatric evaluation. “Ameliorating” also includes shortening or reduction in duration of a symptom.

As used herein, “Reducing incidence” of symptoms or adverse events associated with blood infusion during the treatment of diseases.

The “subject” or “individual” refers to animal, mammals, or human subject. In some embodiments, mammal is preferably a human. Mammals also include, but are not limited to, farm animals, sport animals, pets, primates, horses, dogs, cats, mice and rats.

As used herein, “pharmaceutically acceptable carrier” includes any material, which when combined with the conjugate retains the conjugate's activity and is non-reactive with the subject's immune systems. Examples include, but are not limited to, any of the standard pharmaceutical carriers such as a phosphate buffered saline solution, water, emulsions such as oil/water emulsion, and various types of wetting agents. Compositions comprising such carriers are formulated by well-known conventional methods. Exemplary carriers are hypertonic sodium chloride and isotonic sodium chloride (e.g., phosphate buffered saline). Hypertonic and isotonic carriers are of use in formulating deoxygenated PEGylated of the invention (e.g., carbon monoxide bound iron, and unbound iron) and PEGylated of the invention in which the iron atom is bound to oxygen.

The term “about” as used herein, when referring to a measurable value is meant to encompass variations of ±10%, preferably ±5%, more preferably ±1% and still more preferably ±0.1% from the specified value.

In one aspect, the invention provides a method for treating or preventing at least one symptom, such as cardiovascular events, necrosis, hypoxia, myocardial infraction, sickle cell anaemia, haemorrhagic shock, organ injury such as brain or kidney injury in an individual comprising administering to the individual an effective amount of pegylated hemoglobin in at least two cycles.

In another aspect, the invention provides a method for ameliorating, controlling, reducing incidence of, or delaying the development or progression of at least one symptom, such as cardiovascular events, necrosis, hypoxia, myocardial infraction, sickle cell anaemia, haemorrhagic shock, organ injury such as brain or kidney injury in an individual comprising administering to the individual an effective amount of pegylated hemoglobin in at least two cycles.

In some embodiments, the present invention discloses a process for improved infusion of PEGylated HB-CO (PEG-HB-CO) in a subject in need thereof, the process comprising:
a. infusing first cycle of PEGylated HB-CO (PEG-HB-CO) in the subject; and
b. infusing second cycle of PEGylated HB-CO (PEG-HB-CO in the subject,
wherein, the subject is intravenously and/or subcutaneously infused with PEG-Hb-CO in at least two cycles, and
wherein the infusion process is safe to subject; wherein the infusion process improves one or more conditions selected from tissue oxygenation, maintains blood pressure, and controls adverse effect of hypervolemia in the subject.

In some embodiments, the present invention discloses a process for improved infusion of PEGylated HB-CO (PEG-HB-CO) in the subject in need thereof, the process comprising:
a. infusing first cycle of PEGylated HB-CO (PEG-HB-CO) in the subject at a suitable flow rate; and
b. infusing second cycle of PEGylated HB-CO (PEG-HB-CO) in the subject at a suitable flow rate,
wherein the PEG-HB-CO is administered in at least 2 cycles,
wherein the first cycle of PEG-HB-CO has a higher flow rate than the second cycle, and
wherein the first cycle delivers a lower amount of PEG-HB-CO than the second cycle.

In some embodiments, the present invention discloses an infusion process comprising PEG-HB-CO, wherein the subject is not able to maintain tissue oxygenation because of obstruction in a blood vessel.

In some embodiments, the present invention discloses a process for improved infusion of PEGylated HB-CO (PEG-HB-CO), wherein the process maintains the tissue oxygenation in the subject.

In some embodiments, the present invention discloses a process for improved infusion of PEGylated HB-CO (PEG-HB-CO) in the subject, wherein the infusion process improves patient safety.

In some embodiments, the present invention discloses a process for improved infusion of PEGylated HB-CO (PEG-HB-CO) in the subject, wherein patient safety is improved by improving one of more conditions selected from tissue oxygenation, maintains blood pressure, and controls adverse effect of hypervolemia in the subject.

In some embodiments, the present invention discloses a process for improved infusion of PEGylated HB-CO (PEG-HB-CO) in the subject, wherein the patient safety is improved and there is no toxicity observed in the subject.

In some embodiments, the present invention discloses a process for improved infusion of PEGylated HB-CO (PEG-HB-CO) in the subject, wherein the patient safety is improved and there is no death observed in the subject.

In yet another embodiment, the present invention discloses a process for improved infusion of PEGylated HB-CO (PEG-HB-CO) in the subject, wherein the infusion process controls adverse effects of hypervolemia.

In another embodiment, the present invention discloses a process for improved infusion of PEGylated HB-CO (PEG-HB-CO) in the subject, wherein the infusion process maintains the blood pressure of the subject.

In another embodiment, the present invention discloses a process for improved infusion of PEGylated HB-CO (PEG-HB-CO) in the subject, wherein the blood pressure is maintained in clinically acceptable range.

In some embodiments, the present invention discloses a process for improved infusion of PEGylated HB-CO (PEG-HB-CO), wherein the process maintains the tissue oxygenation in the subject.

In some embodiments, the present invention discloses a process of reducing the incidence and/or alleviating one or more symptoms characterized by the lack of blood supply in a subject, the process comprising:
a. infusing first cycle of PEGylated HB-CO (PEG-HB-CO) in the subject; and
b. infusing second cycle of PEGylated HB-CO (PEG-HB-CO) in the subject,
wherein, the subject is administered with a therapeutically effective amount of PEG-HB-CO in at least two cycles.

The present invention provides an improved infusion process of PEGylated HB-CO in the subject in need thereof comprising,
a. infusing PEGylated HB-CO in suitable amount and at suitable flow rate in first cycle; and
b. infusing PEGylated HB-CO in suitable amount and at suitable flow rate in second cycle; wherein the first cycle has higher flow rate than the second cycle.

In some embodiments, the present invention discloses a process of reducing the incidence and/or alleviating one or more symptoms characterized by the lack of blood supply in a subject, the process comprising:
a. infusing first cycle of PEGylated HB-CO (PEG-HB-CO) in the subject; and
b. infusing second cycle of PEGylated HB-CO (PEG-HB-CO) in the subject;
wherein the subject is administered with a therapeutically effective amount of PEG-HB-CO in at least two cycles.

In some embodiments, the present invention discloses an improved infusion process of PEGylated-HB-CO (PEG-HB-CO) in the subject in need thereof, the process comprising:
a. infusing first cycle of PEGylated HB-CO (PEG-HB-CO) in the subject; and
b. infusing second cycle of PEGylated HB-CO (PEG-HB-CO) in the subject,
wherein the subject is intravenously and/or subcutaneously infused with PEG-Hb-CO in at least two cycles.

In some embodiments, the present invention discloses an improved infusion process of PEGylated HB-CO in the subject in need thereof comprising:
a. infusing PEGylated HB-CO in suitable amount and at suitable flow rate in first cycle; and
b. infusing PEGylated HB-CO in suitable amount and at suitable flow rate in second
cycle; wherein the first cycle has higher flow rate than the second cycle.

In another embodiment, the present invention discloses an improved infusion process of PEGylated HB-CO in the subject, wherein the first cycle has higher flow rate at least by about 5%, about 10%, about 15%, about 20%, about 25%, about 30%, about 35%, about 40%, about 45%, about 50%, about 55%, about 60%, about 65%, about 70%, about 75%, about 80%, about 90%, about 95% and about 100% than the second cycle.

In some embodiments, he present invention discloses an improved infusion process of PEGylated HB-CO in the subject, wherein the first cycle has higher flow rate at least by about 20%, about 25%, about 30%, about 35%, about 40%, about 45%, about 50%, about 55%, about 60%, about 65%, about 70%, about 75%, and about 80%, than the second cycle.

In some embodiments, the present invention discloses an improved infusion process of PEGylated HB-CO in the subject, wherein the first cycle has higher flow rate at least by about 40%, about 45%, about 50%, about 55%, and about 60% than the second cycle.

In some embodiments, the present invention discloses an improved infusion process of PEGylated HB-CO in the subject, wherein the first cycle has higher flow rate at least by about 40%, about 45%, and about 50%, than the second cycle.

The present invention provides an improved infusion process of PEGylated hemoglobin in the subject in need thereof wherein the subject having condition is an anemia selected from the group consisting of blood loss anemias, anemias caused by faulty red blood cell production, anemias caused by red blood cell destruction, and a combination thereof.

In some embodiments, wherein said anemia is sickle cell disease.

The present invention provides an improved infusion process of PEGylated hemoglobin in the subject in the need thereof comprising,
a. infusing PEGylated HB-CO in suitable amount and at suitable flow rate in first cycle; and
b. infusing PEGylated HB-CO in suitable amount and at suitable flow rate in second cycle,
wherein the first cycle delivers lower amount of infused PEGylated hemoglobin than the second cycle.

In such embodiment, the first cycle delivers lower amount of pegylated hemoglobin at least by about 5%, about 10%, about 15%, about 20%, about 25%, about 30%, about 35%, about 40%, about 45%, about 50%, about 55%, about 60%, about 65%, and about 70% than second or subsequent cycle.

In another embodiment, the present invention discloses an improved infusion process of PEGylated HB-CO in the subject, wherein the first cycle delivers a lower amount of pegylated hemoglobin at least about 20%, about 25%, about 30%, about 35%, about 40%, about 45%, about 50%, about 55%, about 60%, about 65%, and about 70% than the second or subsequent cycle.

In another embodiment, the present invention discloses an improved infusion process of PEGylated HB-CO in the subject, wherein the first cycle delivers a lower amount of pegylated hemoglobin at least about 50%, about 55%, about 60%, about 65%, and about 70% than the second or subsequent cycle.

In another embodiment, the present invention discloses an improved infusion process of PEGylated HB-CO in the subject, wherein the first cycle delivers a lower amount of pegylated hemoglobin at least about 60%, about 65%, and about 70% than the second or subsequent cycle.

In some embodiments, the first cycle has about 100% increase in the infusion rate than the second cycle. In some embodiments, the first cycle has about 90% increase in the infusion rate than the second cycle. In some embodiments, the first cycle has about 80% increase in the infusion rate than the second cycle. In some embodiments, the first cycle has about 70% increase in the infusion rate than the second cycle. In some embodiments, the first cycle has about 60% increase in the infusion rate than the second cycle. In some embodiments, the first cycle has about 50% increase in the infusion rate than the second cycle. In some embodiments, the first cycle has about 40% increase in the infusion rate than the second cycle. In some embodiments, the first cycle has about 30% increase in the infusion rate than the second cycle. In some embodiments, the first cycle has about 20% increase in the infusion rate than the second cycle. In some embodiments, the first cycle has about 10% increase in the infusion rate than the second cycle.

In some embodiments, the first cycle has an increased infusion rate from about 10% to about 100% than the second cycle.

In some embodiments, the present invention discloses an infusion process of PEGylated HB-CO in the subject, wherein the first cycle administers about 40% of the total dose and wherein the second cycle administers about 60% of the total dose.

In some embodiments, the present invention discloses an improved infusion process of administering PEGylated HB-CO (PEG-HB-CO) in the subject in need to treat at least one symptom characterized by the lack of blood supply in the subject, the process comprising:
a. infusing therapeutically effective amount of PEG-Hb-CO in the subject in first cycle; and
b. infusing therapeutically effective amount of PEG-Hb-CO in the subject in second cycle,
wherein the subject is intravenously and/or subcutaneously infused with PEG-Hb-CO in at least two cycles,
wherein, the first cycle has a flow rate about 5% to about 100% higher than the second cycle, or about 20% to 80% higher than the second cycle,
wherein, the first cycle delivers about 5% to about 70% or about 20% to about 70% or about 50% to about 70% lower amount of infused PEG-Hb-CO than second cycle, and
wherein, the process of PEGylated HB-CO (PEG-HB-CO) infusion does not lead to a transient increase in the blood pressure with such a level which is clinically unacceptable.

In some embodiments, the present invention discloses an improved infusion process of administering PEGylated HB-CO (PEG-HB-CO), wherein the subject is administered with therapeutically effective amount of PEG-HB-CO for about 2 hours to about 8 hours in at least 2 cycles.

In some embodiments, the present invention discloses an improved infusion process of administering PEGylated HB-CO (PEG-HB-CO), wherein the subject is administered with therapeutically effective amount of PEG-HB-CO for about 2 hours in at least 2 cycles.

In some embodiments, the present invention discloses an improved infusion process of administering PEGylated HB-CO (PEG-HB-CO), wherein the first cycle delivers PEG-HB-CO in the range of about 5 ml/kg, about 10 ml/kg, about 15 ml/kg, about 20 ml/kg, about 25 ml/kg, about 30 ml/kg, about 35 ml/kg, about 40 ml/kg, about 45 ml/kg, about 50 ml/kg, about 55 ml/kg, about 60 ml/kg, about 65 ml/kg, about 70 ml/kg, about 75 ml/kg, about 80 ml/kg, about 85 ml/kg, about 90 ml/kg, about 95 ml/kg, about 100 ml/kg, about 105 ml/kg, about 110 ml/kg, about 115 ml/kg, about 120 ml/kg, about 125 ml/kg, about 130 ml/kg, about 135 ml/kg, about 140 ml/kg, about 145 ml/kg, about 150 ml/kg, about 155 ml/kg, about 160 ml/kg, about 165 ml/kg, about 170 ml/kg, about 175 ml/kg, about 180 ml/kg, about 185 ml/kg, about 190 ml/kg, and about 195 ml/kg, about 200 ml/kg, about 225 ml/kg, and about 250 ml/kg wherein the total blood volume is at least 500 ml.

In some embodiments, the present invention discloses an improved infusion process of administering PEGylated HB-CO (PEG-HB-CO), wherein the first cycle delivers PEG-HB-CO in the range of about 100 ml/kg, about 105 ml/kg, about 110 ml/kg, about 115 ml/kg, about 120 ml/kg, about 125 ml/kg, about 130 ml/kg, about 135 ml/kg, about 140 ml/kg, about 145 ml/kg, about 150 ml/kg, about 155 ml/kg, about 160 ml/kg, about 165 ml/kg, about 170 ml/kg, about 175 ml/kg, about 180 ml/kg, about 185 ml/kg, about 190 ml/kg, and about 195 ml/kg, about 200 ml/kg wherein the total blood volume is at least 500 ml.

In one embodiment, the maximum first cycle does not exceed 50 ml/kg, preferably does not exceed 100 ml/kg, and more preferably does not exceed 200 ml/kg for the first cycle.

In some embodiments, the present invention discloses an improved infusion process of administering PEGylated HB-CO (PEG-HB-CO), wherein the first cycle lasts for at least 5 minutes, about 10 minutes, about15 minutes, about 20 minutes, about 25 minutes, about 30 minutes, and about 35 minutes.

In some embodiments, the present invention discloses an improved infusion process of administering PEGylated HB-CO (PEG-HB-CO), wherein the first cycle lasts for at least about 15 minutes, about 20 minutes, about 25 minutes, about 30 minutes, and about 35 minutes.

In some embodiments, the present invention discloses an improved infusion process of administering PEGylated HB-CO (PEG-HB-CO), wherein the first cycle lasts for at least about 30 minutes.

In some embodiments, the present invention discloses an improved infusion process of administering PEGylated HB-CO (PEG-HB-CO), wherein the flow rate maintained during first cycle is at least by 3 ml/min, about 4 ml/min, about 5 ml/min, about 6 ml/min, about 6.5 ml/min, about 7 ml/min, 8 ml/min based on the weight of subject and effective amount of pegylated hemoglobin needs to be administrated.

In some embodiments, the present invention discloses an improved infusion process of administering PEGylated HB-CO (PEG-HB-CO), wherein the flow rate maintained during first cycle is about 4 ml/min, about 5 ml/min, about 6 ml/min, about 6.5 ml/min, about 7 ml/min based on the weight of subject and effective amount of pegylated hemoglobin needs to be administrated.

In some embodiments, the flow rate is maintained during first cycle is at least by 6.7 ml/min.

In some embodiments, the flow rate is maintained during first cycle is dependent on the weight of subject and effective amount of pegylated hemoglobin administered to the subject.

In some embodiments, the present invention discloses an improved infusion process of administering PEGylated HB-CO (PEG-HB-CO), wherein the second cycle delivers PEG-HB-CO in the range of about 201 ml/kg to about 500 ml/kg, wherein the total blood volume is at least 500 ml.

In some embodiments, the present invention discloses an improved infusion process of administering PEGylated HB-CO (PEG-HB-CO), wherein the second cycle delivers PEG-HB-CO in the range of about 201 ml/kg, about 205 ml/kg, about 210 ml/kg, about 215 ml/kg, about 220 ml/kg, about 225 ml/kg, about 230 ml/kg, about 235 ml/kg, about 240 ml/kg, about 250 ml/kg, about 300 ml/kg, about 350 ml/kg, about 355 ml/kg, about 360 ml/kg, about 365 ml/kg, about 370 ml/kg, about 375 ml/kg, about 380 ml/kg, about 385 ml/kg, about 390 ml/kg, about 395 ml/kg, about 400 ml/kg, about 405 ml/kg, about 410 ml/kg, about 415 ml/kg, about 420 ml/kg, about 425 ml/kg, about 430 ml/kg, about 435 ml/kg, about 440 ml/kg, about 445 ml/kg, about 450 ml/kg, about 455 ml/kg, about 460 ml/kg, about 465 ml/kg, about 470 ml/kg, about 475 ml/kg, about 480 ml/kg, and about 485 ml/kg, about 490 ml/kg, about 495 ml/kg, and about 500 ml/kg.

In some embodiments, the present invention discloses an improved infusion process of administering PEGylated HB-CO (PEG-HB-CO), wherein the second cycle delivers PEG-HB-CO in the range of about 275 ml/kg, about 300 ml/kg, about 350 ml/kg, about 355 ml/kg, about 360 ml/kg, about 365 ml/kg, about 370 ml/kg, about 375 ml/kg, about 380 ml/kg, about 385 ml/kg, about 390 ml/kg, about 395 ml/kg, about 400 ml/kg, about 405 ml/kg, about 410 ml/kg, about 415 ml/kg, about 420 ml/kg, about 425 ml/kg, about 430 ml/kg, about 435 ml/kg, about 440 ml/kg, about 445 ml/kg, about 450 ml/kg, about 455 ml/kg, about 460 ml/kg, about 465 ml/kg, about 470 ml/kg, about 475 ml/kg, about 480 ml/kg, and about 485 ml/kg, about 490 ml/kg, about 495 ml/kg, and about 500 ml/kg.

In some embodiments, the present invention discloses an improved infusion process of administering PEGylated HB-CO (PEG-HB-CO), wherein the second cycle delivers PEG-HB-CO in the range of about 300 ml/kg, about 350 ml/kg, about 355 ml/kg, about 360 ml/kg, about 365 ml/kg, about 370 ml/kg, about 375 ml/kg, about 380 ml/kg, about 385 ml/kg, about 390 ml/kg, about 395 ml/kg, and about 400 ml/kg.

In some embodiments, the dosing regimen comprises administering a dose of about 8 ml/kg in two cycles, if subject weight is about 62.5 kg, he needs 500 ml pegylated hemoglobin needs to be administrated. First cycle will have 200 ml pegylated hemoglobin to be administrated, followed by second cycle which will have remaining 300 ml pegylated hemoglobin needs to be administrated.

In some embodiments, the present invention discloses an improved infusion process of administering PEGylated HB-CO (PEG-HB-CO), wherein the flow rate maintained during second cycle is about 10 min, about 20 min, about 30 min, about 40 min, about 50 min, about 60 min, about 70 min, about 80 min, about 90 min and about 100 min.

In some embodiments, the present invention discloses an improved infusion process of administering PEGylated HB-CO (PEG-HB-CO), wherein the flow rate maintained during second cycle is about 30 min, about 40 min, about 50 min, about 60 min, about 70 min, about 80 min, about 90 min.

In some embodiments, the flow rate is maintained during second cycle is at least by 1 ml/min, about 2 ml/min, about 2.5 ml/min, about 3 ml/min, about 3.3 ml/min, about 4 ml/min, about 4.5 ml/min based on the weight of subject and effective amount of pegylated hemoglobin needs to be administrated.

In some embodiments, the flow rate maintained in second cycle will always be lower than flow rate maintained in first cycle.

In some embodiments, the present invention discloses an improved infusion process of PEGylated HB-CO (PEG-HB-CO) in the subject in the need to reduce the incidence and/or alleviate at least one of the adverse effects selected from the following:
a. transient increase in the blood pressure with such a level which is clinically unacceptable;
b. cardiovascular events;
c. haemorrhagic shock;
d. intracranial haemorrhage;
e. hypoxia;
f. necrosis;
g. myocardial injury;
h. acute myocardial infarction;
i. organ injury such as brain or kidney injury.
j. heart rate, respirations, and temperature;
k. hypertension;
l. pulmonary hypertension;
m. ischemic stroke;
   wherein the subject is intravenously and/or subcutaneously infused with PEG-HB-CO in at least two cycles,
   wherein the flow rate in the first cycle is about 5% to about 100% or about 20% to about 80% or about 40% to about 60% higher than the second cycle, and
   wherein the first cycle delivers a lower amount of pegylated hemoglobin at least about 5%, to about 70% than the second or subsequent cycle.

In some embodiments, the present invention discloses an improved infusion process of administering PEGylated HB-CO (PEG-HB-CO) in the subject in need to treat at least one symptom characterized by the lack of blood supply in the subject, the process comprising:
a. infusing therapeutically effective amount of PEG-Hb-CO in the subject in first cycle; and
b. infusing therapeutically effective amount of PEG-Hb-CO in the subject in second cycle,
wherein the subject is intravenously and/or subcutaneously infused with PEG-Hb-CO in at least two cycles,
wherein the first cycle has a flow rate about 5% to about 100% higher than the second cycle, or about 20% to 80% higher than the second cycle,
wherein the first cycle delivers about 5% to about 70% or about 20% to about 70% or about 50% to about 70% lower amount of infused PEG-Hb-CO than second cycle, and
wherein the process of PEGylated HB-CO (PEG-HB-CO) infusion does not lead to a transient increase in the blood pressure with such a level which is clinically unacceptable.

In some embodiments, the present invention discloses a method for treating a subject suffering from inadequate supply of blood to a tissue or organs; the method comprising;
a. administering the PEGylated HB-CO (PEG-HB-CO) in the subject through infusion; and
b. improving the subject condition,
wherein the infusion is performed in at least two cycles, and
wherein the subject is administered with a therapeutically effective amount of PEG-HB-CO in at least two cycles.

In some embodiments, the present invention discloses a method for administering a PEGylated HB-CO (PEG-HB-CO) in the subject comprising;
a. infusing therapeutically effective amount of PEG-Hb-CO in the subject in first cycle; and
b. infusing therapeutically effective amount of PEG-Hb-CO in the subject in second cycle,
wherein the subject is intravenously and/or subcutaneously infused with PEG-Hb-CO in at least two cycles, and
wherein the administration is safe to subject; wherein the administration is useful in the treatment of a diseases condition.

In some embodiments, the intravenous infusion of PEGylated hemoglobin can be
administrated to a subject before standard of care.

In some embodiments, the intravenous infusion of PEGylated hemoglobin can be administrated to a subject before the administration of recombinant tissue plasminogen activator (rtPA), or mechanical thrombectomy.

In some embodiments, the intravenous infusion of PEGylated hemoglobin sufficient enough to deliver enough oxygen to target tissues which is ischemic or suffering from ischemia.

In some embodiments, the invention provides a method for reducing incidence of or treating at least one symptom in an individual, comprising administering to the individual an effective amount of pegylated hemoglobin wherein said pegylated hemoglobin is perfused in individual in at least two cycles.

The present invention provides an improved infusion process of PEGylated hemoglobin comprising:
a. infusing PEGylated hemoglobin in suitable amount and at suitable flow rate in first cycle; and
b. infusing PEGylated hemoglobin in suitable amount and at suitable flow rate in second cycle,
wherein the intravenous infusion of PEGylated hemoglobin does not lead to transient increase in the blood pressure with such a level which is clinical unacceptable.

The present invention provides an improved infusion process of PEGylated hemoglobin comprising,
a. infusing PEGylated hemoglobin in suitable amount and at suitable flow rate in first cycle; and
b. infusing PEGylated hemoglobin in suitable amount and at suitable flow rate in second cycle,
wherein the intravenous infusion of PEGylated hemoglobin can be administrated to a subject before standard of care.

The present invention provides an improved infusion process of PEGylated hemoglobin comprising,
a. infusing PEGylated hemoglobin in suitable amount and at suitable flow rate in first cycle; and
b. infusing PEGylated hemoglobin in suitable amount and at suitable flow rate in second cycle,
wherein the intravenous infusion of PEGylated hemoglobin can be administrated to a subject before the administration of recombinant tissue plasminogen activator (rtPA), or mechanical thrombectomy.

The present invention provides an improved infusion process of PEGylated hemoglobin comprising,
a. infusing PEGylated hemoglobin in suitable amount and at suitable flow rate in first cycle; and
b. infusing PEGylated hemoglobin in suitable amount and at suitable flow rate in second cycle,
wherein the intravenous infusion of PEGylated hemoglobin sufficient enough to deliver enough oxygen to target tissues which is ischemic or suffering from ischemia.

The pegylated hemoglobin can be administered to an individual via any suitable route preferably by intravenous route. The pegylated hemoglobin can be administered with pharmaceutically acceptable vehicles such as saline, Ringer's solution, dextrose solution, and the like. The particular dosage regimen, i.e., dose, timing and repetition, will depend on the particular individual and that individual's medical history.

In some embodiments, the subject is selected from animal, mammals, or human subject.

In some embodiments, the subject is human.

In some embodiments, an improved infusion process of administering PEG-HB-CO to a subject, wherein the PEG-HB-CO is administered in the subject for about 2 hours, about 3 hours, about 4 hours, about 5 hours, about 6 hours, about 7 hours, and about 8 hours in at least 2 cycles. In yet another embodiment, the present invention discloses an improved infusion process of administering PEG-HB-CO to a subject, wherein PEG-HB-CO is administered in the subject for about 2 hours in at least 2 cycles.

The skilled in the art would appreciate the selection of suitable dose based on the weight of the subject which requires scientific experiment to determine the therapeutic effect of the dose. The invention provides the selection of the subject having weight selected from about 5 kg to about 62.5 kg.

In some embodiments, an improved infusion process of administering PEG-HB-CO to a subject, wherein the PEG-HB-CO is administered in dose-dependent manner, wherein PEG-HB-CO dose is dependent on the subject body weight.

In some embodiments, the dose amount can vary if subject weight is above about 62.5 kg. In another embodiment, the dose amount will remain same if subject weight is above about 62.5 kg.

In some embodiments, the body weight of human subject is selected from about 5 kg, about 5 kg, about 6 kg, about 7 kg, about 8 kg, about 9 kg, about 10 kg, about 11 kg, about 12 kg, about 13 kg, about 14 kg, about 15 kg, about 16 kg, about 17 kg, about 18 kg, about 19 kg, about 20 kg, about 21 kg, about 22 kg, about 23 kg, about 24 kg, about 25 kg, about 26 kg, about 27 kg, about 28 kg, about 29 kg, about 30 kg, about 31 kg, about 32 kg, about 33 kg, about 34 kg, about 35 kg, about 36 kg, about 37 kg, about 38 kg, about 39 kg, about 40 kg, about 41 kg, about 42 kg, about 43 kg, about 44 kg, about 45 kg, about 46 kg, about 47 kg, about 48 kg, about 49 kg, about 50 kg, about 51 kg, about 52 kg, about 53 kg, about 54 kg, about 55 kg, about 56 kg, about 57 kg, about 58 kg, about 59 kg, about 60 kg, about 61 kg, about 62 kg and 62.5 kg and above.

In some embodiments, an improved infusion process of administering PEG-HB-CO to a subject, wherein the PEG-HB-CO dose in the dose dependent manner is selected from about 300 mg/kg to about 20,000 mg/kg.

In some embodiments, an improved infusion process of administering PEG-HB-CO to a subject, wherein the subject body weight is 62.5 and above; wherein the dose is administrated to the subject is about 20,000 mg/kg.

In some embodiments, the dosing regimen comprises administering a dose of about 8 ml/kg in two cycles, if the subject weight is about 62.5 kg, he needs 500 ml pegylated hemoglobin needs to be administrated. First cycle will have 200 ml pegylated hemoglobin to be administrated, followed by second cycle which will have remaining 300 ml pegylated hemoglobin needs to be administrated.

If subject weight is more than 62.5 kg, he needs 500 ml pegylated hemoglobin needs to be administrated. However, in absence of desire result the dose administration can be extended to 20 g/kg.

However, other dosage regimens may be useful, depending on the pattern of pharmacokinetic decay that the practitioner wishes to achieve. For example, in some embodiments, dosing from can have more than two cycles. In some embodiments the dosing can be made in three cycles or four cycles.

In some embodiments, dosing cycles can be performed continually one after another or intermittently with suitable time interval.

In some embodiments, the present invention discloses an improved infusion process of administering PEGylated HB-CO (PEG-HB-CO) in the subject, wherein the process maintains the blood pressure in the subject in clinically acceptable range.

In some embodiments, the present invention discloses an improved infusion process, wherein process exhibits improvement by maintaining systolic pressure in the clinically acceptable range between the range of 120 mm Hg to about 180 mm Hg.

In yet another embodiment, the present invention discloses an improved infusion process, wherein process exhibits improvement by maintaining diastolic pressure in the clinically acceptable range below 95 mm Hg.

In some embodiments, the present invention discloses an improved infusion process of administering PEGylated HB-CO (PEG-HB-CO) in the subject in need to treat at least one symptom characterized by the lack of blood supply in the subject, the process comprising:
a. infusing first cycle of PEGylated HB-CO (PEG-HB-CO) in the subject; and
b. infusing second cycle of PEGylated HB-CO (PEG-HB-CO) in the subject,
wherein the process exhibits improvement by obtaining systolic pressure in the clinically acceptable range between the range of 120 mm Hg to about 180 mm Hg, and
wherein the process exhibits improvement by obtaining diastolic pressure below 95 mm Hg.

In some embodiments, the present invention discloses an improved infusion process of administering PEGylated HB-CO (PEG-HB-CO) in the subject, wherein the process reduces the stroke risk measured by National Institutes of Health Stroke Scale (NIHSS) score.

In some embodiments, the present invention discloses an improved infusion process of administering PEGylated HB-CO (PEG-HB-CO) in the subject, wherein the process exhibits improvement by reducing National Institutes of Health Stroke Scale (NIHSS) score.

In some embodiments, wherein the process exhibits improvement by obtaining the stroke risk between about 0 to about 16 NIHSS score.

In some embodiments, the subject NIHSS score is analysed on day 0 or 1 on day 90.

In some embodiments, the subject mRS score is analysed on day 0 or 1 on day 90.

In one aspect, the invention provides a method for treating or preventing at least one symptom or disorder characterized by ischemia in an individual comprising administering to the individual an effective amount of pegylated hemoglobin in at least two cycles.

In one aspect, the invention provides a method for treating or preventing at least one symptom or disorder characterized by blood pressure in an individual comprising administering to the individual an effective amount of pegylated hemoglobin in at least two cycles.

In one aspect, the invention provides a method for treating or preventing at least one symptom or disorder characterized by stroke in an individual comprising administering to the individual an effective amount of pegylated hemoglobin in at least two cycles.

Assessment and treatment should not be delayed for stroke it is a medical emergency where even a single minute can make a difference. In some embodiments, the intravenous infusion of PEGylated hemoglobin according to the invention has the advantage of being convenient for the subject and health care professionals, it can use as primary health care treatment.

In one aspect, the invention provides a method for treating or preventing at least one symptom or disorder characterized by lack of consciousness in an individual comprising administering to the individual an effective amount of pegylated hemoglobin in at least two cycles.

In one aspect, the invention provides a method for treating or preventing at least one symptom or disorder characterized by myocardial infarction in an individual comprising administering to the individual an effective amount of pegylated hemoglobin in at least two cycles.

In some embodiments, the myocardial infarction present when the troponin values (cTn) with at least one value above the 99th percentile upper reference limit (URL). The myocardial injury is considered acute if there is a rise and/or fall of cTn values.

In some embodiments, the present invention discloses a process sufficient oxygen delivery to target tissues which is ischemic or suffering from ischemia is achieved by the administration of about 5 ml/kg to about 500 ml/kg dose of PEG-Hb-CO.

In some embodiments, sufficient oxygen delivery to target tissues which is ischemic or suffering from ischemia is achieved by the administration of maximum 500 ml/kg dose of PEGylated hemoglobin for the treatment regimen or until suppression of disease symptoms is achieved.

In some embodiments, where the subject's tolerance to the PEGylated hemoglobin is unknown, the first dose is administered by intravenous infusion till the dose will reach 200 ml/kg, followed by second dose administer by intravenous infusion till the second dose reached 500 ml/kg if the subject's tolerance for the PEGylated hemoglobin is acceptable.

In some embodiments, administration is by intravenous administration, preferably intravenous infusion.

In some embodiments, the intravenous infusion of PEG-Hb-CO, can administrated to a subject before standard of care for example before the administration of recombinant tissue plasminogen activator (rtPA), or mechanical thrombectomy.

In some embodiments, the intravenous infusion of PEGylated hemoglobin does not in leads to fluid overload in a human patient.

In some embodiments, the intravenous infusion of PEGylated hemoglobin does not lead to transient increase in the blood pressure while simultaneously time improves the blood flow.

In some embodiments, the intravenous infusion of PEGylated hemoglobin improves the cerebral blood flow.

In some embodiments, the intravenous infusion of PEGylated hemoglobin uses in the treatment selected from hypovolemia, anaemia, ischemia and hypoxia.

In some embodiments, the intravenous infusion of PEGylated hemoglobin uses in the treatment selected from Leg ulcer, burns and Necrosis.

In some embodiments, the intravenous infusion of PEGylated hemoglobin does not result in cardiovascular events.

In some embodiments, the intravenous infusion of PEGylated hemoglobin uses in the treatment selected from stroke, MI, osteonecrosis, splenic rupture, pulmonary hypertension and retinal disease and sickle cell anaemia.

In some embodiments, the intravenous infusion of PEGylated hemoglobin does not result in brain injury.

In some embodiments, the intravenous infusion of PEGylated hemoglobin uses in the treatment of brain injury.

In some embodiments, the intravenous infusion of PEGylated hemoglobin uses in the treatment of kidney injury.

In some embodiments, the intravenous infusion of PEGylated hemoglobin does not result in kidney injury.

In some embodiments, the intravenous infusion of PEGylated hemoglobin does not result in Haemorrhagic shock.

In some embodiments, any volume repletion therapy must be stopped prior to infusion of PEG-Hb-CO.

In some embodiments, the infusion should be followed by a normal saline flush (0.9% sodium chloride).

In some embodiments, the present invention discloses a method of treating and/or alleviating oxygen deprived brain tissue in proximity to a blood vessel occlusion. The occlusion being a blood clot primarily classified as a thrombus or embolism. The region of deprivation being identified as the infarct on radiological imaging with an ischemic core. The peri-infarct regions defined with perfusion imaging identifying hypoxic, but salveagble tissue called the penumbra. Treating this region with an agent that can reduce penumbra hypoxia levels including perfusion through collaterals, dissolution of clot by intravenous thrombolytic/fibrinolytics or removal through a catheter directed mechanical device, the method comprising:
a. identifying stroke by radiological imaging Computed Tomography or Magnetic Resonance methods;
b. additional use of contrast agents for perfusion testing to delineate the ischemic core and the peri-infarct hypoxic penumbra;
c. use of algorithms, imaging scoring systems including Alberta stroke programme early CT score (ASPECTS) or others used by those skilled in the art to further identify non-salveagable to salveagable differences through mismatch scoring, collateral scores or comparable methods;
d. administering to the subject a therapeutically effective amount of PEGylated HB-CO (PEG-HB-CO);
e. additionally treating the subject with Intravenous thrombolytics, mechanical thrombectomy or combinations to facilitate rapid and sustained clot removal and penumbra reperfusion;
f. using angiography including CTA or MRA to guide retrieval and removal of clot;
g. short-term outcome efficacy measures are determined by patient functional scoring from baseline, post-procedure times including short term 24 h and days up to 7;
h. quantitative measurements including one or more such as Glasgow Coma scale, Barthel Index, the National Institutes of Health Stroke Scale or similar;
i. application of monitoring early score changes to determine probability of long-term outcome;
j. further use a disability score including modified Rankin scores throughout the patient triage and treatment including 90 d outcomes as durable recovery,
wherein the PEG-HB-CO is administered in at least 2 cycles,
wherein the first cycle of PEG-HB-CO has a higher flow rate than the second cycle,
wherein the first cycle delivershas a lower amount of infused PEG-HB-CO than the second cycle,
wherein PEG-HB-CO infusion schedule are selected from 30 minute to 120 minute schedule given in ≥1 administration in the dose dependent manner are selected from about 160 mg/mL, to about 320 mg/mL, and
wherein the method reduces or alleviates ASPECTS, NIHSS and mRS scores, achieving improved recovery with improved functions and reduced disability.

### Examples

The following Examples are provided for illustration purpose and scope of the invention should not be considered limiting with the teaching of the examples. The Examples enable that two cycle infusion of PEG-HB-CO is safe for the subject and improves the disease condition. Wherein the disease conditions can be selected from Sickle Cell disease, anemia, cardiovascular events, haemorrhagic shock, intracranial haemorrhage, hypoxia, necrosis, myocardial injury, acute myocardial infarction, organ injury such as brain or kidney injury, heart rate, respirations, and temperature, hypertension, pulmonary hypertension.

### Example 1. Two-Phase Infusion for Subjects with Large Vessel Occlusion Anterior Acute Ischemic Stroke

In this study 47 subjects enrolled in the clinical trial. The trial was conducted to evaluate the effects of PEG-Hb-CO (PP-007) on acute ischemic stroke subjects in combination with standard of care treatment.

Fast infusion of an agent that can improve cerebral blood flow is anticipated to have a positive effect on the ischemic core and hypoxic penumbra. Human albumin infusion formulations have been used for a variety of clinical conditions and was previously studied in several human AIS clinical trials. Specifically, the ALIAS trials used 2 g/kg of 25% IV solution (~500-600 mL) administered over 2 hours showed limited functional benefits. In contrast, safety findings included increased rates of symptomatic intracranial haemorrhage (sICH), volume overload and pulmonary oedema were observed. Albumin is not recommended in AIS, and any use requires strict blood pressure (BP) monitoring requirements.

The HEMERA-1 study enrolled 47 subjects that were eligible for standard of care interventions outlined in the 2019 AHA/ISC guidelines. Briefly, a confirmed non-haemorrhagic stroke, presenting within 3.5-4 h (IVT eligibility) and 24 h (MT eligibility) from last known well were eligible. Subjects deemed by investigators as not likely to survive the study period (90 d), with severely compromised cardiopulmonary functions and those with poor neurological function preceding the stroke were excluded.

PP-007 has the unique capability to improve perfusion with limited impacts on BP as compared to albumin. Therefore, the infusion rate must be strongly considered to ensure safety and efficacy. Specifically, injured neurons can release tissue plasminogen activator protein that has been shown to increase risk of sICH. This risk can be elevated with treatment intervention including a large volume parenteral drug like rapidly administered albumin pushing high pressures onto the injured and potentially constricted blood vessel walls. Conversely, PP-007 has the capability to deliver O2 to the injured area and dramatically slowing the infusion rate could allow injured neurons to die and reduce good outcomes. A further challenge is maintaining adequate blood pressure levels ensuring good pressures needed to maintain perfusion and brain oxygenation with too low or too high equally associated with poor outcomes and safety.

A split rate infusion protocol has been developed to enhance early O2 transfer to hypoxic neurons at a level sufficient to provide favourable clinical outcomes without increasing the risk of sICH. Clinical measurements including BP are captured in real-time during infusion periods to evaluate the effects of BP on achieving both good short and long-term outcomes (i.e. National Institutes of Health Stroke Scale 24 h and day 7; modified Rankin scores of 0-2 at day 90, respectively).

PP-007 (40 mg/mL, 500 mL 20 g total Hb protein) was first infused at a higher rate of 6.67 ml/min for 30 minutes. This resulted in 40% of the total dose (8000 mg) being administered rapidly. The second, slower rate was then started at 3.33 ml/min for 90 minutes. This resulted in the remaining 60% of the total dose (12,000 mg) to be administered slowly (Table 1). This two-rate infusion protocol was intended to achieve rapid, early perfusion and oxygenation of the infarcted region with the second slower rate maintaining the 02 steady-state.

The same dose of PEG-HB-CO was again repeated in two infusion cycles on day 2 (Table 1).

Stroke non-contrast computer tomography (NCCT) imaging is used to develop an ALBERTA STROKE Program Early CT Score (ASPECTS) aiding the treatment decisions for AIS based on brain injury scores (10-0 with higher scores less injury). For example, MT is currently only recommended for ASPECTS≥6 with lower score treatments associated with increased sICH and poor outcomes.

A second dose of PP-007 was administered at the same High/slow infusion rate was added to achieve benefits were there is an unmet need and with lower risk of sICH. Bp was monitored across both treatment times and used to evaluate the safety and effectiveness of PP-007 (table 2 and Table 3) in a wide range of stroke presentations including posterior, non-large vessel occlusion, large vessel occlusion in differing locations.

The split High/slow infusion process was shown to result in good NIHSS and mRS scores (versus medical management or historical controls) as well as showing no incidence of sICH across 47 subjects enrolled in the HEMERA-1 clinical trial. No sICH was observed across the PP-007 treatment groups, whether in combination with IVT, or MT or both treatments (Table 4).

The outcome of Day 1 and Day 2 infusion of PEG-HB-CO in two cycles is given in table 4. Table 4 shows that subject #001-301 administered with PEG-HB-CO in two cycles on day 1 and day 2, wherein the flow rate of first cycle is 6.67 ml/min and the dose volume is 200 ml/kg, wherein the infusion rate of second cycle is 3.33 ml/min and the infusion dose volume is 300 ml/kg. The subject exhibits improvement and shows NIHSS score 0 on day 7 representing 0 risk of Ischemic stroke in the subject. Subject also shows 1 mRS score on day 90 representing no significant risk of disability in subject.

Subject #001-304 was administered with PEG-HB-CO in two cycles on day 1 and day 2, wherein the flow rate of first cycle is 6.67 ml/min and the dose volume is 200 ml/kg, wherein the infusion rate of second cycle is 3.33 ml/min and the infusion dose volume is 300 ml/kg. The subject exhibits improvement and shows 1 NIHSS score on day 7 representing minor risk of ischemic stroke in the subject. Further, the subject also shows 0 mRS score on day 90 representing no risk of disability in subject.

Subject #003-313 was also administered with PEG-HB-CO in two cycles on day 1 and day 2, wherein the flow rate of first cycle is 6.67 ml/min and the dose volume was 200 ml/kg, wherein the infusion rate of second cycle was 3.33 ml/min and the infusion dose volume was 300 ml/kg. The subject improvement shows 0 NIHSS score on day 7 representing no risk of ischemic stroke in the subject. Further, the subject also shows 0 mRS score on day 90 representing no risk of disability in subject.

TABLE 1
Two-phase infusion for Subjects administered with PEG-HB-CO
Trial - 1 (PIS007-101, HEMERA-1)

| | Rate | | | Dose | % of total |
|---|---|---|---|---|---|
| | ml/min | Min | ml/kg | in mg | dose |
| 1^{st} dose | 6.7 ml/min | 30 minutes | 200 ml/kg | 8000 | 40 |
| 2^{nd} dose | 3.3 ml/min | 90 minutes | 300 ml/kg | 12,000 | 60 |

TABLE 2
Depicts the blood pressure obtained during two-phase infusion of infusing
PEG-HB-CO on day 1 with both the cycles having dose as mentioned in Table 1

| | DAY 1 PP-007 Dose #1 | | | | | | | | | |
|---|---|---|---|---|---|---|---|---|---|---|
| | | | Firs cycle | | Second cycle | | | | | |
| | Blood | | 15 | 30 | 45 | 60 | 1 hr 15 | 1 hr 30 | 1 hr 45 | |
| Subject | Pressure | Baseline | mins ± | mins ± | mins ± | mins ± | mins ± | mins ± | mins ± | 2 hr ± |
| ID | Measure | 1 | 5 mins | 5 mins | 5 mins | 5 mins | 5 mins | 5 mins | 5 mins | 5 mins |
| #001- | Systolic | 177 | 134 | 132 | 134 | 142 | 130 | 157 | 140 | NP |
| 301 | Diastolic | 75 | 82 | 79 | 95 | 88 | 63 | 72 | 87 | |
| #001- | Systolic | 155 | 119 | 130 | 116 | 117 | 112 | 94 | 112 | 148 |
| 304 | Diastolic | 109 | 72 | 75 | 89 | 80 | 77 | 68 | 78 | 79 |
| #003- | Systolic | 136 | 159 | 160 | 147 | 138 | 136 | 134 | 143 | 146 |
| 313 | Diastolic | 80 | 87 | 81 | 72 | 72 | 81 | 73 | 75 | 74 |

TABLE 3
Depicts the blood pressure obtained during two-phase infusion of infusing
PEG-HB-CO on day 1 with both the cycles having dose as mentioned in table 1

| | DAY 2 PP-007 Dose #2 | | | | | | | | | |
|---|---|---|---|---|---|---|---|---|---|---|
| | | | First cycle | | Second cycle | | | | | |
| | Blood | | 15 | 30 | 45 | 60 | 1 hr 15 | 1 hr 30 | 1 hr 45 | |
| Subject | Pressure | Baseline | mins ± | mins ± | mins ± | mins ± | mins ± | mins ± | mins ± | 2 hr ± |
| ID | Measure | 1 | 5 mins | 5 mins | 5 mins | 5 mins | 5 mins | 5 mins | 5 mins | 5 mins |
| #001- | Systolic | 145 | 131 | 128 | 126 | 120 | 122 | 116 | 115 | 113 |
| 301 | Diastolic | 76 | 70 | 68 | 65 | 61 | 60 | 59 | 60 | 61 |
| #001- | Systolic | 157 | 168 | 172 | 172 | 169 | 169 | 170 | 137 | 136 |
| 304 | Diastolic | 89 | 92 | 90 | 90 | 95 | 95 | 90 | 82 | 78 |
| #003- | Systolic | 115 | 126 | 128 | 140 | 133 | 140 | 122 | 135 | 129 |
| 313 | Diastolic | 65 | 87 | 80 | 73 | 78 | 70 | 101 | 72 | 66 |

TABLE 4
Day 1 and Day 2 Two-phase infusion of PEG-HB-CO Outcomes

| | Outcome measures | | | | | |
|---|---|---|---|---|---|---|
| | NIHSS (Early function) | | | mRS (Long-term disability) | | |
| Subject | BASE- | 24 | day | BASE- | DAY | DAY |
| ID | LINE | h | 7 | LINE | 30 | 90 |
| #001- | 16 | 3 | 0 | 0 | 0 | 1 |
| 301 | | | | | | |
| #001- | 13 | 1 | 1 | 0 | 0 | 0 |
| 304 | | | | | | |
| #003- | 8 | 0 | discharged | 0 | 0 | NP |
| 313 | | | early | | | |
| #003- | 20 | 18 | 16 | 0 | 4 | 3 |
| 311 | | | | | | |

We claim:

1. A process for improved infusion of PEGylated HB-CO (PEG-HB-CO) in a subject in need thereof, the process comprising:
a. infusing a first cycle of PEGylated HB-CO (PEG-HB-CO) in the subject; and
b. infusing a second cycle of PEGylated HB-CO (PEG-HB-CO) in the subject;
wherein the subject is intravenously and/or subcutaneously infused with PEG-HB-CO in at least two cycles;
wherein the first cycle of PEG-HB-CO has a higher flow rate than the second cycle;
wherein the first cycle delivers a lower amount of PEG-HB-CO than the second cycle; and
wherein the infusion process is safe to the subject; wherein the infusion process improves one or more conditions selected from tissue oxygenation, maintains blood pressure, and controls adverse effect of hypervolemia in the subject.

2. The process as claimed in claim 1, wherein the subject is not able to maintain tissue oxygenation because of obstruction in a blood vessel;
wherein the process maintains the tissue oxygenation in the subject;
wherein the infusion process improves patient safety, and wherein the infusion process controls adverse effects of hypervolemia, and maintains the blood pressure of the subject.

3. The process as claimed in claim 1, wherein the subject is human.

4. A process of reducing the incidence and/or alleviating one or more symptoms characterized by the lack of blood supply in a subject, the process comprising:
a. infusing a first cycle of PEGylated HB-CO (PEG-HB-CO) in the subject; and
b. infusing a second cycle of PEGylated HB-CO (PEG-HB-CO) in the subject,
wherein the subject is administered with a therapeutically effective amount of PEG-HB-CO in at least two cycles.

5. The process of reducing the incidence and/or alleviating one or more symptoms as claimed in claim 4, wherein the symptoms are selected from the group consisting of:
a. transient increase in the blood pressure with such a level which is clinically unacceptable;
b. cardiovascular events;
c. haemorrhagic shock;
d. intracranial haemorrhage;
e. hypoxia;
f. necrosis;
g. myocardial injury;
h. acute myocardial infarction;
i. organ injury such as brain or kidney injury;
j. heart rate, respirations, and temperature;
k. hypertension; and
l. pulmonary hypertension,
wherein the subject is intravenously and/or subcutaneously infused with PEG-HB-CO in at least two cycles,
wherein the flow rate in the first cycle is about 5% to about 100% or about 20% to about 80% or about 40% to about 60% higher than the second cycle, and
wherein the first cycle has a lower amount of pegylated hemoglobin at least about 5%, to about 70% than the second or subsequent cycle;
wherein the process of PEGylated HB-CO (PEG-HB-CO) infusion does not lead to a transient increase in the blood pressure with such a level which is clinically unacceptable.

6. An improved infusion process of administering PEGylated HB-CO (PEG-HB-CO) in a subject in need thereof to treat at least one symptom characterized by the lack of blood supply in the subject, the process comprising:
a. infusing a first cycle of PEGylated HB-CO (PEG-HB-CO) in the subject; and
b. infusing a second cycle of PEGylated HB-CO (PEG-HB-CO) in the subject,
wherein the process maintains the blood pressure in the subject in clinically acceptable range;
wherein the process exhibits improvement by obtaining systolic pressure in the clinically acceptable range between the range of 120 mm Hg to about 180 mm Hg, and
wherein the process exhibits improvement by obtaining diastolic pressure below 95 mm Hg.

7. The process as claimed in claim 1, wherein the first cycle has a lower amount of pegylated hemoglobin at least about 5%, to about 70% than the second or subsequent cycle, or at least by about 20%, to about 70% than the second or subsequent cycle, or at least by about 50%, to about 70%, than the second or subsequent cycle.

8. The process as claimed in claim 4, wherein the first cycle has a lower amount of pegylated hemoglobin at least about 5%, to about 70% than the second or subsequent cycle, or at least by about 20%, to about 70% than the second or subsequent cycle, or at least by about 50%, to about 70%, than the second or subsequent cycle.

9. The process as claimed in claim 6, wherein the first cycle has a lower amount of pegylated hemoglobin at least about 5%, to about 70% than the second or subsequent cycle, or at least by about 20%, to about 70% than the second or subsequent cycle, or at least by about 50%, to about 70%, than the second or subsequent cycle.

10. The process as claimed in claim 1, wherein the first cycle is maintained for at least 5 minutes to at least about 35 minutes; wherein the first cycle is maintained for at least 20 minutes to about at least 30 minutes;
wherein the second cycle is maintained for at about 10 minutes to about 100 minutes, or at about 30 minutes to about 90 minutes.

11. The process as claimed in claim 1, wherein the first cycle maintains flow rate at about 3 ml/min to about 8 ml/min or about 4 ml/min to about 7 ml/min, preferably at about 6.7 ml/min;
wherein the second cycle maintains a flow rate of about 1 ml/min to about 4.5 ml/min, or about 2 ml/min to about 3.5 ml/min, preferably 3.3 ml/min.

12. The process as claimed in claim 4, wherein the first cycle maintains flow rate at about 3 ml/min to about 8 ml/min or about 4 ml/min to about 7 ml/min, preferably at about 6.7 ml/min;
wherein the second cycle maintains a flow rate of about 1 ml/min to about 4.5 ml/min, or about 2 ml/min to about 3.5 ml/min, preferably 3.3 ml/min.

13. The process as claimed in claim 6, wherein the first cycle maintains flow rate at about 3 ml/min to about 8 ml/min or about 4 ml/min to about 7 ml/min, preferably at about 6.7 ml/min;
wherein the second cycle maintains a flow rate of about 1 ml/min to about 4.5 ml/min, or about 2 ml/min to about 3.5 ml/min, preferably 3.3 ml/min.

14. The process as claimed in claim 1, wherein the first cycle has a therapeutic effective amount of PEG-HB-CO administered in the range of 5 ml/kg to about 225 ml/kg, or in the range of 100 ml/kg to about 200 ml/kg;
wherein the second cycle has a therapeutic effective amount of PEG-HB-CO administered in the range of 200 ml/kg to about 500 ml/kg, or in the range of about 300 ml/kg to about 400 ml/kg;
wherein the PEG-HB-CO is administered in the subject for about 2 hours to about 8 hours in at least 2 cycles.

15. The process as claimed in claim 4, wherein the first cycle has a therapeutic effective amount of PEG-HB-CO administered in the range of 5 ml/kg to about 225 ml/kg, or in the range of 100 ml/kg to about 200 ml/kg;
wherein the second cycle has a therapeutic effective amount of PEG-HB-CO administered in the range of 200 ml/kg to about 500 ml/kg, or in the range of about 300 ml/kg to about 400 ml/kg;
wherein the PEG-HB-CO is administered in the subject for about 2 hours to about 8 hours in at least 2 cycles.

16. The process as claimed in claim 6, wherein the first cycle has a therapeutic effective amount of PEG-HB-CO administered in the range of 5 ml/kg to about 225 ml/kg, or in the range of 100 ml/kg to about 200 ml/kg;
wherein the second cycle has a therapeutic effective amount of PEG-HB-CO administered in the range of 200 ml/kg to about 500 ml/kg, or in the range of about 300 ml/kg to about 400 ml/kg;
wherein the PEG-HB-CO is administered in the subject for about 2 hours to about 8 hours in at least 2 cycles.

17. The process as claimed in claim 1, wherein the PEG-HB-CO is administered in a dose-dependent manner; wherein PEG-HB-CO dose is dependent on the subject body weight, the PEG-HB-CO dose in the dose dependent manner is selected from about 300 mg/kg to about 20,000 mg/kg, preferably the dose administered to the subject is about 20,000 mg/kg;
wherein the subject is administered with PEG-HB-CO before receiving standard of care;
wherein the standard of care is selected from intravenous thrombolytics including recombinant tissue plasminogen activator (rtPA), mechanical thrombectomy, bone marrow transplants, administering supplemental oxygen and/or mechanical ventilation.

18. The process as claimed in claim 4, wherein the PEG-HB-CO is administered in a dose-dependent manner; wherein PEG-HB-CO dose is dependent on the subject body weight, the PEG-HB-CO dose in the dose dependent manner is selected from about 300 mg/kg to about 20,000 mg/kg, preferably the dose administered to the subject is about 20,000 mg/kg;
wherein the subject is administered with PEG-HB-CO before receiving standard of care;
wherein the standard of care is selected from intravenous thrombolytics including recombinant tissue plasminogen activator (rtPA), mechanical thrombectomy, bone marrow transplants, administering supplemental oxygen and/or mechanical ventilation.

19. The process as claimed in claim 6, wherein the PEG-HB-CO is administered in a dose-dependent manner; wherein PEG-HB-CO dose is dependent on the subject body weight, the PEG-HB-CO dose in the dose dependent manner is selected from about 300 mg/kg to about 20,000 mg/kg, preferably the dose administered to the subject is about 20,000 mg/kg;
wherein the subject is administered with PEG-HB-CO before receiving standard of care;
wherein the standard of care is selected from intravenous thrombolytics including recombinant tissue plasminogen activator (rtPA), mechanical thrombectomy, bone marrow transplants, administering supplemental oxygen and/or mechanical ventilation.

20. The process as claimed in claim 1, wherein the subject is selected from animal, mammals, or human subject.

21. The process as claimed in claim 4, wherein the subject is selected from animal, mammals, or human subject.

22. The process as claimed in claim 6, wherein the subject is selected from animal, mammals, or human subject.

23. The process as claimed in claim 1, wherein the process reduces the stroke risk measured by National Institutes of Health Stroke Scale (NIHSS) score; wherein the process exhibits improvement by obtaining the stroke risk between about 0 to about 16 NIHSS score;
wherein the subject NIHSS score is analysed on day 0, day 1, or on day 90;
wherein the process reduces the risk of disability in the subject measured by Modified Rankin Scale (mRS); wherein the process exhibits improvement by obtaining the disability risk between about 0 to about 3 mRS score;
wherein the subject mRS score is analysed on day 0, day 1, or on day 90.

24. The process as claimed in claim 4, wherein the process reduces the stroke risk measured by National Institutes of Health Stroke Scale (NIHSS) score; wherein the process exhibits improvement by obtaining the stroke risk between about 0 to about 16 NIHSS score;
wherein the subject NIHSS score is analysed on day 0, day 1, or on day 90;
wherein the process reduces the risk of disability in the subject measured by Modified Rankin Scale (mRS); wherein the process exhibits improvement by obtaining the disability risk between about 0 to about 3 mRS score;
wherein the subject mRS score is analysed on day 0, day 1, or on day 90.

25. The process as claimed in claim 6, wherein the process reduces the stroke risk measured by National Institutes of Health Stroke Scale (NIHSS) score; wherein the process exhibits improvement by obtaining the stroke risk between about 0 to about 16 NIHSS score;
wherein the subject NIHSS score is analysed on day 0, day 1, or on day 90;
wherein the process reduces the risk of disability in the subject measured by Modified Rankin Scale (mRS); wherein the process exhibits improvement by obtaining the disability risk between about 0 to about 3 mRS score;
wherein the subject mRS score is analysed on day 0, day 1, or on day 90.

26. The process as claimed in claim 1, wherein the first cycle administers about 40% of the total dose and wherein the second cycle administers about 60% of the total dose;
wherein the first cycle administers 8,000 mg of the dose and wherein the second cycle administers 12,000 mg of the dose.

27. The process as claimed in claim 4, wherein the first cycle administers about 40% of the total dose and wherein the second cycle administers about 60% of the total dose;
wherein the first cycle administers 8,000 mg of the dose and wherein the second cycle administers 12,000 mg of the dose.

28. The process as claimed in claim 6, wherein the first cycle administers about 40% of the total dose and wherein the second cycle administers about 60% of the total dose;
wherein the first cycle administers 8,000 mg of the dose and wherein the second cycle administers 12,000 mg of the dose.
